SEQUENCE LISTING (13 sequences)

SEQ ID NO: 1 - protein, 93 aa, synthetic construct
WKQNKDGIWYKAEHASFTVTAPEGIITRYKGPWTGHPQAGVLQKGQTIKYDEQKFDGHVWVSWETFEGETVYMPVRTWDAKTGKVGKLWGEIK

SEQ ID NO: 2 - DNA, 285 bp, synthetic construct
  source 1..285: mol_type = other DNA
tggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 3 - protein, 486 aa, synthetic construct
MLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 4 - protein, 498 aa, synthetic construct
MRGSHHHHHHGSMLTAIDYLTKKGWKISSDPRTYDGYPKNYGYRNYHENGINYDEFCGGYHRAFDVYSNETNDVPAVTSGTVIEANDYGNFGGTFVIRDANDNDWIYGHLQRGSMRFVVGDKVNQGDIIGLQGNSNYYDNPMSVHLHLQLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 5 - DNA, 1461 bp, synthetic construct
  source 1..1461: mol_type = other DNA
atgctaactgctattgactatcttacgaaaaaaggttggaaaatatcatctgaccctcgcacttacgatggttaccctaaaaactacggctacagaaattaccatgaaaacggcattaattatgatgagttttgtggtggttatcatagagcttttgatgtttacagtaacgaaactaacgacgtgcctgctgttactagcggaacagttattgaagcaaacgattacggtaattttggtggtacattcgttattagagacgctaacgataacgattggatatatgggcatctacaacgtggctcaatgcgatttgttgtaggcgacaaagtcaatcaaggtgacattattggtttacaaggtaatagcaactattacgacaatcctatgagtgtacatttacatttacaattacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 6 - protein, 140 aa, synthetic construct
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGY

SEQ ID NO: 7 - DNA, 420 bp, synthetic construct
  source 1..420: mol_type = other DNA
gctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatat

SEQ ID NO: 8 - protein, 169 aa, synthetic construct
NKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDG

SEQ ID NO: 9 - DNA, 510 bp, synthetic construct
  source 1..510: mol_type = other DNA
ggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggt

SEQ ID NO: 10 - protein, 505 aa, synthetic construct
AATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 11 - protein, 517 aa, synthetic construct
MRGSHHHHHHGSAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGELLRPKDAKKDEKSQVCSGLAMEKYDITNLNAKQDKSKNGSVKELKHIYSNHIKGNKITAPKPSIQGVVIHNDYGSMTPSQYLPWLYARENNGTHVNGWASVYANRNEVLWYHPTDYVEWHCGNQWANANLIGFEVCESYPGRISDKLFLENEEATLKVAADVMKSYGLPVNRNTVRLHNEFFGTSCPHRSWDLHVGKGEPYTTTNINKMKDYFIKRIKHYYDGGKLEVSKAATIKQSDVKQEVKKQEAKQIVKATDWKQNKDGIWYKAEHASFTVTAPEGIITRYKGPWTGHPQAGVLQKGQTIKYDEVQKFDGHVWVSWETFEGETVYMPVRTWDAKTGKVGKLWGEIK

SEQ ID NO: 12 - DNA, 1518 bp, synthetic construct
  source 1..1518: mol_type = other DNA
gctgcaacacatgaacattcagcacaatggttgaataattacaaaaaaggatatggttacggtccttatccattaggtataaatggcggtatgcactacggagttgatttttttatgaatattggaacaccagtaaaagctatttcaagcggaaaaatagttgaagctggttggagtaattacggaggaggtaatcaaataggtcttattgaaaatgatggagtgcatagacaatggtatatgcatctaagtaaatataatgttaaagtaggagattatgtcaaagctggtcaaataatcggttggtctggaagcactggttattctacagcaccacatttacacttccaaagaatggttaattcattttcaaattcaactgcccaagatccaatgcctttcttaaagagcgcaggatatggaaaagcaggtggtacagtaactccaacgccgaatacaggtgagctcttacgccctaaagacgcaaagaaagatgaaaaatcacaagtatgtagtggtttggctatggaaaaatatgacattacaaatttaaatgctaaacaagataaatcaaagaatgggagcgtgaaagagttgaaacatatctattcaaaccatattaaaggtaacaagattacagcaccaaaacctagtattcaaggtgtggtcatccacaatgattatggtagtatgacacctagtcaatacttaccatggttatatgcacgtgagaataacggtacacacgttaacggttgggctagtgtttatgcaaatagaaacgaagtgctttggtatcatccgacagactacgtagagtggcattgtggtaatcaatgggcaaatgctaacttaatcggatttgaagtgtgtgagtcgtatcctggtagaatctcggacaaattattcttagaaaatgaagaagcgacattgaaagtagctgcggatgtgatgaagtcgtacggattaccagttaatcgcaacactgtacgtctgcataacgaattcttcggaacttcttgtccacatcgttcgtgggacttgcatgttggcaaaggtgagccttacacaactactaatattaataaaatgaaagactacttcatcaaacgcatcaaacattattatgacggtggaaagctagaagtaagcaaagcagcaactatcaaacaatctgacgttaagcaagaagttaaaaagcaagaagcaaaacaaattgtgaaagcaacagattggaaacagaataaagatggcatttggtataaagctgaacatgcttcgttcacagtgacagcaccagagggaattatcacaagatacaaaggtccttggactggtcacccacaagctggtgtattacaaaaaggtcaaacgattaaatatgatgaggttcaaaaatttgacggtcatgtttgggtatcgtgggaaacgtttgagggcgaaactgtatacatgccggtacgcacatgggacgctaaaactggtaaagttggtaagttgtggggcgaaattaaataa

SEQ ID NO: 13 - protein, 12 aa, synthetic construct
MRGSHHHHHHGS

(19) United States
(12) Patent Application Publication
(10) Pub. No.: US 2026/0355089 A1
(43) Pub. Date: Nov. 12, 2026

(54) COSMETIC COMPOSITION COMPRISING AN ENDOLYSIN AND A NONIONIC SURFACTANT COMPRISING A CARBOHYDRATE RESIDUE

(71) Applicant: L'OREAL, Paris (FR)

(72) Inventors: Sylvie JALENQUES, Chevilly Larue (FR); Toni MOINE, Chevilly Larue (FR); Cécilia CLAMENS, SAINT-OUEN (FR); François-Baptiste SCHATZ, Chevilly Larue (FR)

(73) Assignee: L'OREAL, Paris (FR)

(21) Appl. No.: 19/484,732

(22) PCT Filed: May 31, 2024
(86) PCT No.: PCT/EP2024/065106

§ 371 Date: Nov. 14, 2025

(30) Foreign Application Priority Data
Jun. 2, 2023 (FR) 2305535

(51) Int. Cl. A61K 8/66; A61K 8/60; A61K 31/351; A61K 38/16; A61K 47/10; A61K 47/18; A61K 47/20; A61K 47/44; A61P 31/04; A61Q 17/00; A61Q 19/00

(52) U.S. Cl. CPC A61K 8/66; A61K 8/604; A61K 31/351; A61K 38/162; A61K 47/10; A61K 47/18; A61K 47/20; A61K 47/44; A61P 31/04; A61Q 17/00; A61Q 19/00

(57) ABSTRACT

The present invention relates to a composition, particularly a cosmetic composition, comprising, in a physiologically acceptable medium, at least one endolysin, in particular an endolysin derived from a Staphylococcus aureus phage, and at least one nonionic surfactant comprising one or more carbohydrate residue(s).

The invention also relates to the use of such a composition for preventing and/or treating a skin disorder associated with colonization by Staphylococcus aureus in an individual in need thereof, and in particular for preventing and/or treating acne and/or eczema in an individual in need thereof, and to a non-therapeutic cosmetic process for caring for keratin materials, in particular the skin, comprising at least a step of topical application of such a composition to said keratin materials.

### TECHNICAL FIELD

The present invention relates to a composition, particularly a cosmetic composition, comprising, in a physiologically acceptable medium, at least one endolysin, in particular an endolysin derived from a Staphylococcus aureus phage, and at least one nonionic surfactant comprising one or more carbohydrate residue(s).

The invention also relates to the use of a composition of the invention for preventing and/or treating a skin disorder associated with colonization by bacteria of the species Staphylococcus aureus, and in particular for preventing and/or treating acne and/or eczema in an individual in need thereof.

Finally, the invention relates to a non-therapeutic cosmetic process for caring for keratin materials, in particular the skin, comprising the topical application to these keratin materials of a composition according to the invention.

### PRIOR ART

Human skin is permanently populated by a multitude of different microorganisms (bacteria, yeasts and fungi). The resident microbial flora, which is essential to the good health of the skin, consists mainly of propionibacteria (Cutibacterium acnes), staphylococci (Staphylococcus epidermidis and Staphylococcus hominis), corynebacteria and streptococci, and also of a fungal flora composed mainly ofMalassezia.

Certain dermatological disorders are usually due to the disruption of the ecological balance of the resident flora following a preponderant colonization by opportunistic microorganisms that are not beneficial to the skin, such as Staphylococcus aureus, which is known to be associated with atopic dermatitis (eczema), greasy or hyperseborrhoeic skin, and acne.

To counteract this excess colonization by these opportunistic microorganisms that are not beneficial to the skin, it is common practice to use broad-spectrum antimicrobials or bacteriostats. However, the use of these compounds poses the problem of non-specificity of action targeting both the undesirable opportunistic flora and the resident beneficial flora, and the problem of the risk of the appearance of bacterial resistance or imbalances by selecting for resistant bacteria, and also of problems of skin tolerance (irritation, allergies, etc.).

There is thus a need to find novel compounds with good antimicrobial efficacy which do not have the abovementioned drawbacks, and which have good skin tolerance.

It has thus been demonstrated that endolysins from Staphylococcus aureus phages (i.e. phages infecting Staphylococcus aureus) make it possible to specifically target S. aureus and lyse, and thus specifically destroy, this bacterium while preserving the resident skin flora (WO 2012/150858 A1). However, it is well known that one of the main obstacles to the application of endolysins targeting Staphylococcus species is a problem of the stability of these proteins and/or of their enzymatic activity, particularly the maintenance of this activity over time.

Indeed, it has in particular been observed that the lytic action of these enzymes is significantly impaired by interactions with raw materials, in particular when the endolysin is introduced into formulations, particularly cosmetic formulations.

There is thus a need for a surfactant that makes it possible to preserve the antimicrobial activity of the endolysins used in formulations, particularly cosmetic formulations, over time.

There is also a need for a surfactant, the purpose of which is (i) to promote preservation of the antimicrobial activity of the endolysins used in compositions, particularly cosmetic compositions, over time, (ii) while retaining the specificity of these endolysins for the targeted bacteria, in particular Staphylococcus aureus in the present invention.

There is additionally a need for a surfactant, the purpose of which is:
to promote preservation of the antimicrobial activity of the endolysins used in compositions, particularly cosmetic compositions, over time;
while retaining the specificity of these endolysins for the targeted bacteria, in particular Staphylococcus aureus in the present invention; and
while also affording good sensory properties for the composition.

### DISCLOSURE OF THE INVENTION

The aim of the present invention is to solve at least one abovementioned technical problem. Indeed, the inventors have now discovered that caprylyl/capryl glucoside, arachidyl glucoside, and polyglyceryl-3 methylglucose distearate, compared to charged surfactants such as anionic surfactants, amphoteric surfactants and other nonionic surfactants free of carbohydrate residue(s) which are known in the field of formulation, in particular cosmetic formulation, prove able to maintain a good destruction performance of Staphylococcus aureus by the associated endolysin after one week post-preparation of a composition comprising same.

### SUMMARY OF THE INVENTION

As mentioned above, the present invention thus relates to a composition, particularly a cosmetic composition, comprising, in a physiologically acceptable medium:
(i) at least one endolysin; and
(ii) at least one nonionic surfactant comprising one or more carbohydrate residue(s).

As illustrated in the examples below, the Applicant has discovered, surprisingly, that a composition according to the invention comprising an endolysin, in particular an endolysin derived from a Staphylococcus aureus phage, and caprylyl/capryl glucoside, arachidyl glucoside, or polyglyceryl-3 methylglucose distearate, advantageously make it possible to maintain the destruction performance of S. aureus of the endolysin.

Thus, the present invention also relates to the use of a composition of the invention for preventing and/or treating a skin disorder associated with colonization by bacteria of the species Staphylococcus aureus, and in particular for preventing and/or treating acne and/or eczema in an individual in need thereof.

The invention moreover relates to a non-therapeutic cosmetic process for caring for keratin materials, in particular the skin, comprising the topical application to these keratin materials of a composition according to the invention.

### DETAILED DESCRIPTION

For the purposes of the present invention, “surfactant” means any compound which modifies the surface tension between two surfaces. The surfactant compounds are amphiphilic molecules, i.e. they have two parts of different polarity, one part being lipophilic (which retains fatty substances) and apolar, the other part being hydrophilic (miscible or soluble in water) and polar. They thus make it possible to solubilize two immiscible phases by interacting with the apolar phase (i.e. lipophilic, thus hydrophobic) with the hydrophobic part thereof, and the other, polar phase interacts with the hydrophilic part.

“Nonionic surfactant” means a surfactant that has no net charge (that does not ionize in water).

For the purposes of the present invention, “carbohydrate” means a compound of sugar or carbohydrate type, corresponding to a molecule composed essentially of carbon, hydrogen and oxygen atoms.

“Cosmetic” means a composition that is compatible with keratin materials, in particular the skin, mucous membranes and skin appendages, more particularly the skin. The composition according to the invention is non-therapeutic.

“Keratin materials” in particular means the skin, mucous membranes, fibres, eyelashes and skin appendages.

“The skin” means all the skin of the body, and preferably the skin of the face, the scalp, the neckline, the neck, the arms and forearms, the eyelids, around the mouth or behind the ears, the hollow of the elbow, the back of the knees, the hands, the wrists and the ankles, or even more preferably the skin of the face (in particular the forehead, the nose, the cheeks and the chin), the neckline and the neck.

A composition according to the invention comprises a physiologically acceptable medium, i.e. one which has a pleasant colour, odour and feel and which does not give rise to any unacceptable discomfort, i.e. tingling, tightness or redness, that is liable to discourage the user from applying this composition. Needless to say, a person skilled in the art will take care to choose a physiologically acceptable medium such that the advantageous properties of the endolysin(s) of the invention are not, or are not substantially, adversely affected. Thus, by way of illustration, a physiologically acceptable medium may comprise water and/or one or more water-miscible organic solvent(s).

A physiologically acceptable medium according to the invention preferentially has a pH between 4 and 8, more particularly between 4.5 and 7.5. Thus, a composition according to the invention can comprise one or more pH adjuster(s). According to a preferred embodiment, a composition according to the invention comprises at least one pH adjuster.

As used herein, the terms “treat” and “treatment” mean the alleviation of the symptoms associated with a specific disorder or condition and/or the elimination of said symptoms and also the complete disappearance of the disorder or condition in question.

In the context of the present invention, the terms “prevent” and “prevention” mean the reduction, to a lesser degree, of the risk or probability of occurrence of a given phenomenon.

### Endolysin

A composition, particularly a cosmetic composition, according to the invention is first characterized in that it comprises at least one endolysin.

In the embodiments described herein, the endolysin can be a native bacteriophage endolysin or a recombinant endolysin, and can be any endolysin known to those skilled in the art. In the present document, the terms “bacteriophage lysin”, “bacteriophage endolysin” and “endolysin” are used interchangeably. An endolysin can be selected from the group of endolysins defined in documents WO2011/023702, WO2012/146738, WO2003/082184, WO2010/011960, WO2010/149795, WO2010/149792, WO2012/094004, WO2011/023702, WO2011/065854, WO2011/076432, WO2011/134998, WO2012/059545, WO2012/085259, WO2012146738, WO2018/091707, Exebacase™ (Lysin CF-301); SAL200™ or Tonabacase; Auresine™ (Sigma-Aldrich SAE0083), and Ectolysin™ P128.

In the embodiments presented herein, the endolysin is an endolysin specific to Staphylococcus aureus, that is to say that it will effectively lyse Staphylococcus aureus but will not substantially lyse bacteria other than Staphylococcus aureus. In particular, an endolysin used according to the invention will lyse Staphylococcus aureus but not Staphylococcus epidermidis.

The majority of native Staphylococcus bacteriophage endolysins have peptidoglycan hydrolase activity, such as the Ply2638 endolysin which is composed of a C-terminal cell wall binding domain (CBD), a central N-acetylmuramoyl-L-alanine amidase domain, and an N-terminal alanyl-glycyl endopeptidase domain with cysteine, in the case of Ply2638, of an N-terminal alanyl-glycine endopeptidase domain with peptidase_M23 homology, the latter three domains each having peptidoglycan hydrolase activity with distinct target binding specificity and generally being named enzymatically active domains.

The endolysin can be a recombinant endolysin such as a recombinant endolysin specific to Staphylococcus aureus, in particular a chimeric recombinant endolysin specific to Staphylococcus aureus, comprising one or more heterologous domains.

In general, endolysins are constituted of different subunits (domains), for example a cell wall binding domain (CBD) and one or more enzymatic domains having peptidoglycan activity, such as an amidase domain, an M23 domain, and a CHAP (cysteine, histidine-dependent amidohydrolase/peptidase) domain. An example of a chimeric endolysin specific to Staphylococcus aureus comprising one or more heterologous domains is an endolysin comprising an amidase domain from the bacteriophage Ply2638, a lysostaphin M23 domain (S. simulans), and a cell wall binding domain from the bacteriophage Ply2638.

This chimeric endolysin specific to Staphylococcus aureus is a preferred endolysin and is described in detail in the document WO2012/150858. Other preferred endolysins are described in detail in the document WO2013/169104. Other preferred endolysins are described in detail in the document WO2016/142445. Other endolysins preferred according to the invention are fully described in WO2017/046021. Other endolysins preferred according to the invention are fully described in WO2012/146738, WO2003/082184, WO2010/011960, WO2010/149795, WO2011/076432, WO2011/134998, WO2012/085259, WO2012146738 and WO2018/091707.

An endolysin used may comprise a domain having at least 80% sequence identity with a domain described in WO2012/150858, WO2013/169104, WO2016/142445, WO2017/046021, WO2012/146738, WO2003/082184, WO2010/011960, WO2010/149795, WO2011/076432, WO2011/134998, WO2012/085259, WO2012146738 or WO2018/091707.

An endolysin used may have at least 80% sequence identity with an endolysin described in WO2012/150858, WO2013/169104, WO2016/142445, WO2017/046021, WO2012/146738, WO2003/082184, WO2010/011960, WO2010/149795, WO2011/076432, WO2011/134998, WO2012/085259, WO2012146738, WO2018/091707, such as the endolysin with the amino acid sequence of reference SEQ ID NO: 29 in WO2012/150858.

In a particular embodiment, the endolysin is an endolysin derived from a Staphylococcus aureus phage.

For the purposes of the present invention, “endolysin derived from a Staphylococcus aureus phage” means a native or recombinant protein, such as an enzyme or nucleic acid molecule encoding for same, derived from one or more bacteriophage(s) that are capable of lysing the wall of bacteria of the species Staphylococcus aureus.

The endolysin particularly comprises one or more domains for binding to the bacterial wall of Staphylococcus aureus and/or one or more domains for lysing the bacterial wall of Staphylococcus aureus, said binding domain(s) and lysis domain(s) for the bacterial wall of Staphylococcus aureus being derived from one or more identical or different bacteriophage(s) that are capable of lysing the wall of bacteria of the species Staphylococcus aureus.

The endolysin used in the context of the present invention may be in native or recombinant form, in particular in recombinant form.

According to a particular embodiment, the endolysin comprises a first protein sequence comprising a domain for binding to the cell wall of species of the genus Staphylococcus.

In particular, the first protein sequence is derived from the endolysin of the Φ2638a bacteriophage of S. aureus.

For each of the amino acid or nucleic acid sequences of interest, reference sequences are described herein. The present description also encompasses amino acid or nucleic acid sequences (for example enzyme amino acid sequences), having specific percentages of amino acid or nucleotide identity with a reference sequence.

For obvious reasons, throughout the present description, a specific nucleic acid sequence or a specific amino acid sequence which complies, respectively, with the nucleotide or amino acid identity under consideration, must also lead to the production of a protein (or enzyme) which displays the desired biological activity. As used herein, the “percentage identity” between two nucleic acid sequences or between two amino acid sequences is determined by comparing the two optimally aligned sequences through a comparison window.

The portion of the nucleotide or amino acid sequence in the comparison window may thus comprise additions or deletions (e.g. “gaps”) relative to the reference sequence (which does not comprise these additions or deletions) so as to achieve optimal alignment between the two sequences.

The terms “sequence homology” or “sequence identity” or “homology” or “identity” are used interchangeably herein. For the purposes of the invention, this means that in order to determine the percentage of sequence homology or sequence identity of two amino acid sequences or two nucleic acid sequences, the sequences are aligned for optimal comparison.

In order to optimize the alignment between the two sequences, gaps may be introduced in either of the two sequences being compared. This alignment may be performed over the entire length of the sequences being compared. The alignment may also be performed over a shorter length, for example over about twenty, fifty, one hundred or more nucleic acids/bases or amino acids. Sequence identity is the percentage of identical matches between the two sequences over the reported aligned region.

The comparison of sequences and the determination of the percentage of sequence identity between two sequences may be performed using a mathematical algorithm. A person skilled in the art knows that several different computer programs are available for aligning two sequences and determining the identity between two sequences (Kruskal, J. B. (1983) An overview of sequence comparison In D. Sankoff and J. B. Kruskal, (ed.), Time warps, string edits and macromolecules: the theory and practice of sequence comparison, pages 1-44 Addison Wesley).

The percentage of sequence identity between two amino acid sequences or between two nucleotide sequences may be determined using the Needleman-Wunsch algorithm for the alignment of two sequences. (Needleman, S. B. and Wunsch, C. D. (1970) J. Mol. Biol., 48, 443-453). The algorithm allows the alignment of both amino acid sequences and nucleotide sequences. The Needleman-Wunsch algorithm was implemented in the NEEDLE computer program.

For the purposes of the invention, the NEEDLE program of the EMBOSS software package was used (version 2.8.0 or higher, EMBOSS: The European Molecular Biology Open Software Suite (2000) Rice, P. Longden, J. and Bleasby, A. Trends in Genetics 16, (6) pages 276-277, http://emboss.bioinformatics.nl/). For protein sequences, EBLOSUM62 is used for the substitution matrix. For nucleotide sequences, EDNAFULL is used. The optional parameters used are a space opening penalty of 10 and a space extension penalty of 0.5. No end gap penalty is added. In the Output section, Yes has been indicated in response to the question “Brief identity and similarity” and “SRS pairwise” has been indicated as the output alignment format.

After alignment by the NEEDLE program described above, the percentage of sequence identity between a query sequence and a sequence of the invention is calculated as follows: Number of matching positions in the alignment showing an identical amino acid or nucleotide in the two sequences divided by the total length of the alignment after subtracting the total number of gaps in the alignment. The identity defined here may be obtained from NEEDLE using the NOBRIEF option and is labelled in the output of the program as “longest identity”.

The similarity of nucleotide and amino acid sequences, i.e. the percentage identity of the sequences, may be determined by sequence alignments using several other known algorithms, preferably the mathematical algorithm of Karlin and Altschul (Karlin & Altschul (1993) Proc. Natl. Acad. Sci. USA 90: 5873-5877), with hmmalign (HMMER package, http://hmmer.wustl.edu/) or with the CLUSTAL algorithm (Thompson, J. D., Higgins, D. G. & Gibson, T. J. (1994) Nucleic Acids Res. 22, 4673-80) available, for example, at https://www.ebi.ac.uk/Tools/msa/clustalo/or the GAP program (mathematical algorithm of the University of Iowa) or the mathematical algorithm of Myers and Miller (1989—Cabios 4: 11-17) or Clone Manager 9. The preferred parameters used are the default parameters as defined at https://www.ebi.ac.uk/Tools/msa/clustalo/.

The degree of sequence identity (sequence matching) may be calculated using, for example, BLAST, BLAT or BlastZ (or BlastX). A similar algorithm is incorporated in the BLASTN and BLASTP programs of Altschul et al. (1990) J. Mol. Biol., 215, 403-410. BLAST polynucleotide searches are performed with the BLASTN program, score=100, word length=12, so as to obtain polynucleotide sequences homologous to the nucleic acids which encode the protein of interest.

BLAST protein searches are performed with the BLASTP program, score=50, word length=3, to obtain amino acid sequences homologous to the SHC polypeptide. To obtain gapped alignments for comparison, Gapped BLAST is used as described in Altschul et al. (1997) Nucleic Acids Res. 25, 3389-3402. When using the BLAST and Gapped BLAST programs, the default settings of the respective programs are used. Sequence matching analysis may be supplemented by established homology mapping techniques such as Shuffle-LAGAN (Brudno M., Bioinformatics 2003b, 19 Suppl. 1: 154-162) or Markov random fields. Where reference is made to percentages of sequence identity in the present patent application, these percentages are calculated relative to the total length of the longest sequence, unless otherwise indicated.

In particular embodiments, the percentage identity between two sequences is determined using CLUSTAL 0 (version 1.2.4).

Thus, according to a particular embodiment, the first protein sequence comprises a protein sequence comprising at least 80%, in particular at least 90%, more particularly at least 95% sequence identity with the amino acid sequence of reference SEQ ID NO: 1.

“At least 80% sequence identity between two sequences” means that the first sequence may comprise 80%, 81%, 82%, 83%, 84%, 85%, 86%, 87%, 88%, 89%, 90%, 91%, 92%, 93%, 94%, 95%, 96%, 97%, 98%, 99% or 100% sequence identity with the second sequence, whether these sequences are amino acid sequences or nucleic acid sequences. In particular, the first protein sequence consists of an amino acid sequence of reference SEQ ID NO: 1.

The protein sequences described herein may be encoded by one or more allelic variants.

An allelic variant denotes any one of two or more alternative forms of a gene occupying the same chromosomal locus. A preferred nucleic acid variant is a nucleotide sequence that contains one or more silent mutations. Alternatively or in combination, a nucleic acid variant may also be obtained by introducing nucleotide substitutions, which do not result in another amino acid sequence of the polypeptide encoded by the nucleotide sequence, but which correspond to the use of codons of the host organism intended for the production of the polypeptide of the invention. According to a preferred embodiment, a nucleic acid variant encodes a polypeptide that still has its biological function. More preferably, a nucleotide sequence variant encodes a polypeptide displaying binding to the cell wall of species of the genus Staphylococcus, and/or lytic activity. Even more preferably, a nucleic acid variant encodes a polypeptide displaying increased binding to the cell wall of species of the genus Staphylococcus and/or increased lytic activity, as defined hereinbelow. Nucleic acids encoding a polypeptide displaying binding to the cell wall of species of the genus Staphylococcus and/or lytic activity may be isolated from any microorganism.

All these variants may be obtained using techniques known to those skilled in the art, such as library screening by hybridization (Southern blot procedures) under low to medium to high hybridization conditions. Low to medium to high stringency conditions means pre-hybridization and hybridization at 42 °C in 5×SSPE, 0.3% SDS, 200 μg/ml sheared and denatured salmon sperm DNA, and either 25%, 35% or 50% formamide for low to medium to high stringencies, respectively. Next, the hybridization reaction is washed three times for 30 minutes using, for each wash, 2×SSC, 0.2% SDS, and at 55 °C, 65 °C or 75 °C for low to medium to high stringencies.

According to a particular embodiment, the first protein sequence is encoded by a nucleic acid sequence comprising at least 80%, in particular at least 90%, more particularly at least 95% sequence identity with the nucleic acid sequence of reference SEQ ID NO: 2.

In particular, the first protein sequence is encoded by a nucleic acid sequence consisting of a nucleic acid sequence of reference SEQ ID NO: 2.

The binding of a domain to the peptidoglycan cell wall of Staphylococcus genera may be evaluated using assays that are well known to those skilled in the art. In a preferred embodiment, an immunohistochemical technique and/or a gene fusion technique resulting in labelled constructs of either domain are used to evaluate the specific binding of peptides, polypeptides or proteins to the peptidoglycan cell wall of Staphylococcus genera. The signal quantification processes used in the abovementioned immunohistochemical or fusion techniques are well known in the art.

In one embodiment, the binding to the peptidoglycan cell wall of Staphylococcus may be quantified using a fluorescent fusion construct comprising a polypeptide comprising a domain included in a first protein sequence as described previously. Such a cell wall binding assay is described in detail by Loessner et al. (Molecular Microbiology 2002, 44(2): 335-349). In said assay, a solution comprising said fluorescent fusion construct or a negative control, preferably green fluorescent protein (GFP), is subjected to Staphylococcus cells, preferably S. aureus cells, more preferably S. aureus BB255 cells, for a specified period of time, after which the cells are sedimented by centrifugation together with the bound fluorescent fusion constructs. The fluorescent signal of Staphylococcus cells exposed to a fluorescent fusion construct, subtracted from the fluorescent signal of Staphylococcus cells exposed to a negative control, preferably GPF, is a measure of cell binding for the purposes of the present invention.

Examples of evaluation of the binding of an endolysin to the cell wall of species of the genus Staphylococcus which are suitable according to the invention are particularly illustrated in WO 2012/150858 A1.

Preferably, in the context of the present text, a protein sequence will be said to comprise a domain for binding to the peptidogly can cell wall of Staphylococcus genera when, using this assay, an increase in the fluorescent signal of the sedimented cells is detected. The binding is preferably said to be specific. Preferably, an endolysin is described comprising a domain which displays a binding capacity, as defined herein, of at least 50, 60, 70, 80, 90 or 100, 150 or 200% of the peptidoglycan cell wall binding of the endolysin of the Φ2638a bacteriophage of S. aureus (Ply2638) encoded by the nucleic acid sequence of reference SEQ ID NO: 5.

According to a particular embodiment, the binding activity to the cell wall of species of the genus Staphylococcus is measured by an immunohistochemical technique and/or a gene fusion technique, in particular a fluorescent fusion technique, more particularly fusion with a green fluorescent protein.

According to a particular embodiment, the endolysin comprises a protein sequence comprising at least 80%, in particular at least 90%, more particularly at least 95% sequence identity with an amino acid sequence selected from the group consisting of the amino acid sequences of references SEQ ID NO: 3 and SEQ ID NO: 4.

According to a particular embodiment, the endolysin comprises a protein sequence encoded by a nucleic acid sequence comprising at least 80%, in particular at least 90%, more particularly at least 95% sequence identity with the nucleic acid sequence of reference SEQ ID NO: 5.

In particular, the protein sequence may be encoded by a nucleic acid sequence consisting of a nucleic acid sequence of reference SEQ ID NO: 5.

According to a particular embodiment, the endolysin may also comprise a heterologous protein sequence.

“Heterologous protein sequence” means a protein sequence, i.e. an amino acid sequence or a nucleic acid sequence encoding the protein sequence, which is not naturally functionally linked as a neighbouring sequence to said first protein sequence. As used herein, the term “heterologous” may mean “recombinant”. The term “recombinant” refers to a genetic entity different from that generally found in nature. When applied to a nucleotide sequence or nucleic acid molecule, this means that said nucleotide sequence or nucleic acid molecule is the product of various combinations of cloning, restriction and/or ligation steps, and other procedures that result in the production of a construct that is different from a sequence or molecule found in nature.

Such protein or nucleic acid recombination methods are well known to those skilled in the art.

According to a particular embodiment, the endolysin comprises a heterologous protein sequence comprising a lytic domain. Preferably, said lytic domain displays peptidoglycan hydrolase activity.

“Peptidoglycan hydrolase activity”, also defined herein as “lytic activity”, may be evaluated via processes that are well known to those skilled in the art. In one embodiment, the lytic activity may be evaluated spectrophotometrically by measuring the decrease in turbidity of substrate cell suspensions. Preferably, the lytic activity may be evaluated spectrophotometrically by measuring the decrease in turbidity of a suspension of S. aureus, the turbidity being quantified by measuring the OD₅₉₅ spectrophotometrically (Libra S22, Biochrom). More preferably, 200 nM of a polypeptide encoded by a nucleic acid molecule as identified herein are incubated with a suspension of S. aureus having an initial OD₆₀₀ of 1±0.05, as evaluated spectrophotometrically (Libra S22, Biochrom), in PBS buffer pH 7.4, 120 mM sodium chloride for 30 min at 37 °C. The decrease in turbidity is calculated by subtracting the OD₅₉₅ after 30 min of incubation from the OD₅₉₅ before 30 min of incubation.

In the context of the present text, a protein sequence will be said to comprise a lytic domain when, using this assay, a decrease in turbidity of at least 10, 20, 30, 40, 50 or 60% is detected.

Preferably, a decrease of at least 70% is detected. Preferably, an endolysin is described comprising a domain which displays lytic activity of at least 50, 60, 70, 80, 90, 100, 150 or 200% or more of a lytic activity of the endolysin of the Φ2638a bacteriophage of S. aureus (Ply2638) encoded by the nucleic acid sequence of reference SEQ ID NO: 5.

According to a particular embodiment, the lytic activity of the endolysin is measured spectrophotometrically by measuring the decrease in turbidity of a suspension of S. aureus.

In one embodiment, it is possible for the endolysin not to be encoded by an amino acid sequence comprising or consisting of an amino acid sequence selected from the group consisting of the amino acid sequences of references SEQ ID NO: 3 and SEQ ID NO: 4.

In particular, it is possible for the endolysin not to be encoded by a nucleic acid sequence comprising or consisting of the nucleic acid sequence of reference SEQ ID NO: 5, encoding the endolysin of the Φ2638a bacteriophage of S. aureus.

According to a particular embodiment, the heterologous protein sequence comprises a lytic domain, said lytic domain comprising a second and a third protein sequence, said second protein sequence comprising an M23 endopeptidase domain and said third protein sequence comprising an amidase domain.

An endopeptidase domain as used herein preferably cleaves pentaglycine cross-bridges (Trayer, H. R. and Buckley, C. E. (1970) Molecular properties of lysostaphin, a specific bacteriolytic agent for Staphylococcus aureus. J. Biol. Chem. 245, 4842-4846) which are found in the cell wall of Staphylococcus genera, preferably in the cell wall of S. aureus, S. simulans and S. carnosus.

An amidase domain as used herein preferably hydrolyses substrates containing gamma-glutamyl.

The functionality and activity of such domains in a polypeptide may be confirmed by characterizing the cleavage products upon incubation of said polypeptides containing any of these domains with a purified peptidoglycan.

According to a particular embodiment, the endopeptidase and/or amidase activity of the endolysin is measured by characterizing the cleavage products.

According to a particular embodiment, the endopeptidase and/or amidase activity of the endolysin may be measured by measuring the optical density of bacteria in the presence of the endolysin. Such methods are particularly described in Park et al. (Characterization of an endolysin, LysBPS13, from a Bacillus cereus bacteriophage, FEMS Microbiol. Lett. 2012 July; 332(1): 76-83) and in Grishin et al. (A Simple Protocol for the Determination of Lysostaphin Enzymatic Activity, Antibiotics (Basle). 2020 Dec. 17; 9(12): 917).

Preferably, each of the protein sequences and nucleotide sequences encoding the second or third domain is of bacterial or bacteriophage origin.

According to a particular embodiment, said second and third protein sequences are derived, independently of each other, from an enzyme selected from the group consisting of the endolysin of the Φ2638a bacteriophage of S. aureus and the lysostaphin of S. simulans. In particular, one of the second and third protein sequences is derived from the endolysin of the Φ2638a bacteriophage of S. aureus and the other sequence of the second and third protein sequences is derived from the lysostaphin of S. simulans.

According to a particular embodiment, said second protein sequence comprises at least 80%, in particular at least 90%, more particularly at least 95% sequence identity with the amino acid sequence of reference SEQ ID NO: 6 and said third protein sequence comprises at least 80%, in particular 90%, more particularly 95% sequence identity with the amino acid sequence of reference SEQ ID NO: 8.

According to a particular embodiment, said second protein sequence is encoded by a nucleic acid sequence comprising at least 80%, in particular at least 90%, more particularly at least 95% sequence identity with the nucleic acid sequence of reference SEQ ID NO: 7 and said third protein sequence is encoded by a nucleic acid sequence comprising at least 80%, in particular at least 90%, more particularly at least 95% sequence identity with the nucleic acid sequence of reference SEQ ID NO: 9.

According to a particular embodiment, said second protein sequence is encoded by a nucleic acid sequence consisting of the nucleic acid sequence of reference SEQ ID NO: 7 and said third protein sequence is encoded by a nucleic acid sequence consisting of the nucleic acid sequence of reference SEQ ID NO: 9.

According to a particular embodiment, the endolysin comprises a protein sequence comprising at least 80%, in particular at least 90%, more particularly at least 95% sequence identity with an amino acid sequence selected from the group consisting of the amino acid sequences of references SEQ ID NO: 10 and SEQ ID NO: 11.

In particular, the endolysin may comprise a protein sequence consisting of the amino acid sequence of reference SEQ ID NO: 10.

According to a particular embodiment, the endolysin comprises a protein sequence encoded by a nucleic acid sequence comprising at least 80%, in particular at least 90%, more particularly at least 95% sequence identity with a nucleic acid sequence of reference SEQ ID NO: 12. In particular, the endolysin may comprise a protein sequence encoded by a nucleic acid sequence consisting of a nucleic acid sequence of reference SEQ ID NO: 12.

An endolysin comprising a protein sequence encoded by a nucleic acid sequence of reference SEQ ID NO: 12 differs from the endolysin of the Φ2638a bacteriophage of S. aureus in that the N-terminal M23 endopeptidase domain is substituted with an M23 endopeptidase domain from the lysostaphin of S. simulans.

Endolysins that are suitable for use in the invention may be obtained via any method known to those skilled in the art for producing recombinant proteins. In particular, endolysins according to the invention may be obtained by introducing one or more gene(s) of interest, such as the nucleic acid sequences described previously, into the genome of a host organism via a vector.

In another aspect, a nucleic acid construct comprising at least one of the nucleic acid sequences as defined previously is described. This nucleic acid construct may comprise a first nucleic acid sequence encoding a polypeptide comprising a cell wall binding domain, also possibly comprising a second and third nucleic acid sequence as defined previously.

An expression vector comprising such a nucleic acid construct is also described. Preferably, an expression vector comprises a nucleotide sequence as mentioned previously, which is functionally linked to one or more control sequences which direct the production or expression of the encoded polypeptide in a cell, a subject or a cell-free expression system.

An expression vector may be considered a recombinant expression vector. This vector may consist of a plasmid, cosmid, bacteriophage or virus which is transformed by the introduction of a nucleic acid molecule according to the invention. Such transformation vectors according to the host organism to be transformed are well known to those skilled in the art and widely described in the literature.

Another subject described in the present text is a process for transforming host organisms by integration of at least one nucleic acid sequence as described, which transformation may be performed via any suitable means known and widely described in the specialized literature, more particularly via the vector described above.

In another aspect, a cell is described, which comprises a nucleic acid construct or an expression vector as defined previously. A cell may be any prokaryotic or eukaryotic microbial cell which is suitable for expressing an endolysin that is suitable for use in the invention. In a preferred embodiment, said cell is an E. coli cell. In an even more preferred embodiment, said cell is E. coli CL1 blue MRF.

The endolysin obtained may then be purified according to purification methods known in the art such as column chromatography, high performance liquid chromatography, etc.

In a particular embodiment, one or more of the protein sequences as defined in the text may comprise a sequence encoding a tag to facilitate the purification of the resulting endolysin.

Preferably, said tag is chosen from, but is not limited to, a group consisting of a FLAG tag, a poly(His) tag, an HA tag and an Myc tag. More preferably, said tag is a 6×His tag. Even more preferably, said tag is an N-terminal 6×His tag identical to SEQ ID NO: 13.

Endolysins and also methods for producing them are particularly described in patent application WO 2012/150858 A1.

An endolysin, in particular an endolysin derived from a Staphylococcus aureus phage that is suitable for use according to the invention may be present in the composition in freshly prepared form or in lyophilized form.

In the present text, “freshly prepared” is preferably defined as storage for at most 2 days after production at 1.63 mg/mL in lyophilization buffer (50 mM Tris, 500 mM sucrose, 200 mM mannitol, 0.05% polysorbate 20+50% glycerol) at −20 °C followed by thawing immediately prior to evaluating the lytic activity in an assay as identified herein.

“Lyophilized” refers to an endolysin that has been dehydrated by lyophilization, which consists in freezing the protein and then dehydrating it to remove the water.

After lyophilization, an endolysin in lyophilized form may undergo a subsequent reconstitution step by adding water. In one embodiment, the lyophilization and reconstitution may be performed by dialysis against three changes of 300 ml lyophilization buffer (50 mM phosphate or Tris, 500 mM sucrose, 200 mM mannitol, pH 7.4) aliquot and freezing in the gas phase of liquid nitrogen. The lyophilization may be performed under standard conditions, preferably at −40 °C and under vacuum at 75 mTorr for 60 minutes, before increasing the temperature over 5 hours to −10 °C and further increasing over 60 minutes to −10 °C at the same vacuum levels. As a final step, the temperature is preferably increased to °C over 10 hours. The samples are reconstituted by adding water.

A composition according to the invention may comprise a content of endolysin(s), ranging from 0.0001% to 0.10% by weight relative to the total weight of the composition, in particular from 0.0005% to 0.01% by weight relative to the total weight of the composition, more particularly from 0.001% to 0.005% by weight relative to the total weight of the composition.

### Nonionic Surfactant Comprising One or More Carbohydrate Residue(s)

In particular, the carbohydrate residue(s) is (are) monosaccharides comprising 5 to 6 carbon atoms, preferentially selected from glucose, fructose, xylose or galactose, preferably glucose.

In particular, the nonionic surfactant(s) comprising one or more carbohydrate residue(s) is (are) selected from:
i) fatty acid esters of sugar(s) such as fatty acid (poly)glyceryl esters of glucose or alkylglucose having a linear or branched, saturated or unsaturated, C6-22, preferably C16-20, hydrocarbon chain;
ii) ethers of sugar(s) and fatty alcohol(s) such as alkyl(poly)glycosides; and
iii) mixtures thereof.

More particularly, the nonionic surfactant(s) comprising one or more carbohydrate residue(s) is (are) selected from alkyl(poly)glycosides and fatty acid (poly)glyceryl esters of glucose or alkylglucose having a linear or branched, saturated or unsaturated, C6-22, preferably C16-20, hydrocarbon chain, and mixtures thereof.

### Alkyl (Poly)Glycoside

Nonionic surfactants of alkyl(poly)glycoside type are in particular represented by the following general formula (I):

[Chem 1]

R¹O—(R²O)t−(G)v (I)

in which:
R¹ represents a linear or branched alkyl or alkenyl radical including 6 to 24 carbon atoms, particularly 8 to 20 carbon atoms, or an alkylphenyl radical of which the linear or branched alkyl radical includes 6 to 24 carbon atoms, particularly 8 to 20 carbon atoms;
R² represents an alkylene radical including 2 to 4 carbon atoms;
G represents a sugar unit including 5 to 6 carbon atoms;
t denotes a value ranging from 0 to 10, preferably from 0 to 4;
v denotes a value ranging from 1 to 15, preferably from 1 to 4.

According to a particular embodiment, the alkyl(poly)glycoside surfactants are compounds of the formula described above, in which:
R¹ denotes a saturated or unsaturated, linear or branched, alkyl radical including from 8 to 20 carbon atoms,
R² represents an alkylene radical including 2 to 4 carbon atoms,
t denotes a value ranging from 0 to 3 and preferably equal to 0,
G denotes glucose, fructose or galactose, preferably glucose,
it being possible for the degree of polymerization, i.e. the value of v, to range from 1 to 15 and preferably from 1 to 4; the mean degree of polymerization more particularly being between 1 and 2.

The glucosidic bonds between the sugar units are generally of 1-6 or 1-4 type and preferably of 1-4 type.

As examples of alkyl(poly)glycosides, mention may be made of caprylyl/capryl glucoside, such as the product sold under the name Oramix CG 110L® by the company SEPPIC; decyl glucoside sold under the name Sorbithix L-100® by the company Applechem; arachidyl glucoside glucoside, optionally as a mixture with arachidyl alcohol and behenyl alcohol, sold, for example, under the name Montanov 202® by the company SEPPIC; cetylstearyl glucoside, optionally as a mixture with cetylstearyl alcohol, sold, for example, under the name Montanov 68 MB® by the company SEPPIC, under the name Emulgade PL 68/50® by the company BASF and under the name Tego Care CG 90 MB® by the company Evonik Goldschmidt; cocoyl glucoside, such as the product sold under the name Lamesoft P065® by the company BASF; octyldodecyl xyloside, sold, for example, under the name Fluidanov 20× by the company SEPPIC.

The alkyl(poly)glycoside may be used as a mixture with at least one fatty alcohol, particularly a fatty alcohol having from 6 to 24 carbon atoms and more particularly from 8 to 20 carbon atoms.

For example, it is possible to use in combination a fatty alcohol and an alkyl(poly)glycoside, the alkyl part of which is identical to that of the selected fatty alcohol.

The fatty alcohol/alkyl polyglycoside emulsifying mixtures as defined above are known per se. They are described particularly in applications WO92/06778, WO95/13863 and WO98/47610 and prepared according to the preparation processes indicated in these documents.

Among the particularly preferred fatty alcohol/alkyl(poly)glycoside mixtures, mention may be made of the products sold by the company SEPPIC under the name Montanov®, such as the following mixtures:
arachidyl alcohol and behenyl alcohol/arachidyl glucoside—Montanov 202®
cetylstearyl alcohol/cetylstearyl glucoside—Montanov 68 MB®.

In a particular embodiment, the composition according to the invention comprises an alkyl(poly)glycoside surfactant selected from caprylyl/capryl glucoside, arachidyl glucoside and mixtures thereof.

In a particular embodiment, the composition according to the invention does not comprise any decyl glucoside.

### Fatty Acid (Poly)Glyceryl Esters of Glucose or Alkylglucose

The fatty acid (poly)glyceryl esters of glucose or alkylglucose having a linear or branched, saturated or unsaturated C6-C22 hydrocarbon chain are in particular fatty acid (poly)glyceryl esters of alkylglucose having a linear, saturated C6-C22, preferably C16-20, better still C18, hydrocarbon chain.

Among the fatty acid (poly)glyceryl esters of glucose or alkylglucose having a linear or branched, saturated or unsaturated, C6-C22 hydrocarbon chain, polyglyceryl-3-methylglucose distearate is most particularly preferred.

Among the commercial products, mention may be made of the product sold by the company Evonik Goldschmidt under the name Tego Care 450.

The nonionic surfactant(s) comprising one or more carbohydrate residue(s) may be present in the composition according to the invention in a content ranging from 0.01% to 10% by weight, in particular in a content ranging from 0.05% to 8% by weight, more particularly in a content ranging from 0.10% to 5% by weight relative to the total weight of the composition, even more particularly in a content ranging from 0.2% to 3% by weight relative to the total weight of the composition.

A composition according to the invention may comprise one or more endolysin(s) according to the invention and one or more nonionic surfactant(s) comprising one or more carbohydrate residue(s) according to the invention in a mass ratio of endolysin(s)/nonionic surfactant(s) comprising one or more carbohydrate residue(s) of between 0.0001 to 0.5, in particular between 0.0002 and 0.1, more particularly between 0.0004 and 0.05, even more particularly between 0.0006 and 0.02.

### Other Surfactants

The composition according to the invention may contain other surfactant(s), particularly other nonionic surfactants different from those according to the present invention, such as nonionic surfactants selected from C₆-C₃₀ fatty acid alkanolamides, oxyalkylenated fatty alcohols, and mixtures thereof.

C₆-C₃₀ Fatty Acid Alkanolamides

Such surfactants may be selected from monoalkanolamides and dialkanolamides of formula (II):

[Chem 2]

R¹CONR²R³ (II)
in which:
R¹ is a linear or branched, saturated or unsaturated hydrocarbon group having from 6 to 30 carbon atoms,
R² and R³, independently, are hydrogen or a linear or branched, saturated or unsaturated alkanol group having 1 to 10 carbon atoms, with the proviso that only one of R² and R³ is hydrogen.

As examples of surfactants of this type, mention may be made of lauric acid monoethanolamide, lauric acid diethanolamide, lauric acid monopropanolamide, lauric acid monoisopropanolamide, myristic acid monoethanolamide, myristic acid diethanolamide, palmitic acid monoethanolamide, stearic acid monoethanolamide (stearamide MEA), oleic acid monoethanolamide, oleic acid diethanolamide, oleic acid monoisopropanolamide, coconut oil fatty acid monoethanolamide (cocamide MEA), coconut oil fatty acid monopropanolamide, coconut oil fatty acid monoisopropanolamide (cocamide MIPA), erucic acid diethanolamide, palm plant oil fatty acid monoethanolamide, and mixtures thereof.

In particular in formula (II), R¹ is a linear or branched, saturated or unsaturated hydrocarbon group having from 8 to 18 carbon atoms,
R² and R³, independently, are hydrogen or a linear or branched, saturated or unsaturated alkanol group having 2 to 5 carbon atoms, with the proviso that only one of R² and R³ is hydrogen.

More particularly, in formula (II), R² is hydrogen, and R³ is a saturated linear or branched alkanol group having 2 to 5 carbon atoms.

According to a particular embodiment, the appropriate C₆-C₃₀ fatty acid alkanolamide of formula (II) is selected from coconut oil fatty acid monoethanolamide (INCI: cocamide MEA or cocamide monoethanolamine), coconut oil fatty acid monoisopropanolamide (INCI: cocamide MIPA or cocamide monoisopropanolamine), and mixtures thereof.

Such products are commercially available: for example, coconut oil fatty acid monoethanolamide (cocamide MEA) sold under the name Comperlan® 100 by the company Cognis (BASF), coconut oil fatty acid monoisopropanolamide (cocamide MIPA) sold under the trade name Empilan® CIS by the company Innospec Active Chemicals.

According to a particular embodiment, said C₆-C₃₀ fatty acid alkanolamide is cocamide monoisopropanolamine.

The C₆-C₃₀ fatty acid alkanolamide according to the present invention may be present in an amount ranging from 0.5% to 10% by weight, in particular from 0.1% to 5% by weight, relative to the total weight of the composition.

### Oxyalkylated Fatty Alcohols

Oxyalkylenated, in particular oxyethylenated and/or oxypropylenated, alcohols that are preferably used are those that may include from 1 to 150 oxyethylene and/or oxypropylene units, in particular having from 20 to 100 oxyethylene units, in particular fatty alcohols, particularly C₈-C₂₄ and preferably C₁₂-C₁₈ fatty alcohols; these fatty alcohols may or may not be ethoxylated, for instance stearyl alcohol ethoxylated with 20 oxyethylene units (CTFA name Steareth-20), for instance Brij® 78 sold by the company Uniqema, cetearyl alcohol ethoxylated with 30 oxyethylene units (CTFA name Ceteareth-30), cetearyl alcohol ethoxylated with 20 oxyethylene units (CTFA name Ceteareth-20), and the mixture of C₁₂-C₁₅ fatty alcohols including 7 oxyethylene units (CTFA name C₁₂₋₁₅ Pareth-7), for instance the product sold under the name Neodol 25-7® by Shell Chemicals; or in particular oxyalkylenated (oxyethylenated and/or oxypropylenated) alcohols having from 1 to 15 oxyethylene and/or oxypropylene units, in particular ethoxylated C₈-C₂₄ and preferably C₁₂-C₁₈ fatty alcohols, such as stearyl alcohol ethoxylated with 2 oxyethylene units (CTFA name Steareth-2), for instance Brij® 72 sold by the company Uniqema. More particularly, a composition according to the invention also comprises Ceteareth-20.

The oxyalkylenated fatty alcohol may be present in an amount ranging from 0.1% to 10% by weight, in particular from 0.5% to 5% by weight, relative to the total weight of the composition.

A composition according to the invention may comprise at least water, and optionally a water-miscible organic solvent.

In particular, a composition according to the invention may include an amount of water of at least 10% by weight relative to the total weight of the composition, in particular an amount of water ranging from 10% to 98% by weight, more particularly from 20% to 95% by weight, particularly from 30% to 90% by weight and more particularly from 35% to 85% by weight relative to the total weight of the composition.

The water may be sterile demineralized water and/or floral water and/or natural spring or mineral water.

In the present invention, “water-miscible organic solvent” means an organic compound which is liquid at room temperature and the miscibility of which in water is greater than 50% by weight at 25 °C and atmospheric pressure.

The water-miscible organic solvents that may be used in a composition of the invention may also be volatile.

Among the water-miscible organic solvents that may be used in a composition of the invention, mention may be made, for example, of lower monoalcohols having from 2 to 5 carbon atoms, such as ethanol and isopropanol, polyols such as propylene glycol and glycerol.

According to one embodiment, a composition according to the invention may be free of lower monoalcohols having 2 to 5 carbon atoms.

A composition according to the invention may comprise less than 2% by weight of ethanol, preferably less than 1% by weight, more particularly less than 0.5% by weight, relative to the total weight of the composition, in particular less than 0.1% by weight of ethanol, and preferably may be free of ethanol.

### Additional Ingredients

In addition to the abovementioned compounds, a composition according to the invention may of course comprise one or more additional ingredient(s).

Needless to say, a person skilled in the art will take care to select one or more additional ingredient(s) such that the advantageous properties of the endolysin(s) of the invention are not, or are not substantially, adversely affected.

The additional ingredients are present in the compositions in a content which is usual for each of them in a cosmetic composition, in particular in a content which is usual for each of them and which enables them to retain their cosmetic properties, more particularly in a content which is usual for each of them and which enables them, when they are each the only ingredient of a composition according to the invention having this property, to retain their cosmetic property.

Thus, a composition according to the invention may comprise one or more of the following additional ingredient(s), selected from: surfactants; fatty substances; colorants; preserving agents; fragrances; pH adjusters such as organic acids, for instance citric acid; antioxidants; hydrophilic gelling agents such as hydroxypropylmethylcellulose; amino acids such as arginine; carbohydrates; chelating agents; sugar alcohols; cosmetic active agents; and mixtures thereof.

Needless to say, a person skilled in the art will take care to select this or these optional additional ingredient(s), and/or the amount thereof, such that the advantageous properties of a composition according to the invention are not, or are not substantially, adversely affected by the envisioned addition.

The additional ingredient(s) different from those listed below may be present in the composition according to the invention in a concentration of between 0.001% and 20% by weight, in particular from 0.01% to 10% by weight, more particularly between 0.1% and 5% by weight relative to the total weight of the composition.

According to a particular embodiment, a composition according to the invention may comprise at least one additional ingredient selected from an oil, an aromatic alcohol of formula (I), an organic filler, a nonionic surfactant, and mixtures thereof

### Oil

A composition according to the invention may comprise at least one oil.

For the purposes of the present invention, “oil” means a water-immiscible compound which is liquid at 25 °C and atmospheric pressure (1.013×10⁵ Pa).

“Immiscible” means that the mixing of the same amount of water and oil, after stirring, does not result in a stable solution comprising only a single phase, under the abovementioned temperature and pressure conditions. Observation is performed by eye or using a phase-contrast microscope, if necessary, on 100 g of mixture obtained after sufficient stirring with a Rayneri blender to produce a vortex within the mixture (as a guide, 200 to 1000 rpm), the resulting mixture being left to stand, in a closed flask, for 24 hours at room temperature before observation.

“Hydrocarbon oil” means an oil mainly containing hydrogen and carbon atoms and optionally one or more functions selected from hydroxyl, ester, ether and carboxylic functions. A hydrocarbon oil thus consequently does not comprise any silicon or fluorine atoms.

“Silicone oil” refers to an oil comprising at least one silicon atom, and particularly at least one Si—O group, and more particularly an organopolysiloxane.

“Fluoro oil” refers to an oil comprising at least one fluorine atom.

“Apolar hydrocarbon oil” means a hydrocarbon oil comprising only carbon and hydrogen atoms, which is in particular non-aromatic (also called a hydrocarbon).

The term “polar hydrocarbon oil” denotes hydrocarbon oils mainly comprising hydrogen and carbon atoms and one or more functions selected from hydroxyl, ester, ether and carboxylic functions.

In particular, the composition according to the invention may comprise at least one oil selected from volatile and non-volatile oils, in particular with the exception of liquid paraffins.

### Volatile Oils

“Volatile oil” means an oil (or non-aqueous medium) that can evaporate on contact with the skin in less than one hour, at room temperature and at atmospheric pressure. The volatile oil is a volatile cosmetic oil, which is liquid at room temperature, particularly having a non-zero vapour pressure at room temperature and at atmospheric pressure, in particular having a vapour pressure ranging from 0.13 Pa to 40 000 Pa (10-3 to 300 mmHg), in particular ranging from 1.3 Pa to 13 000 Pa (0.01 to 100 mmHg) and more particularly ranging from 1.3 Pa to 1300 Pa (0.01 to 10 mmHg).

According to a particular embodiment of the invention, the volatile oils are such that the flash points are less than 120 °C and the vapour pressure is less than 5 Pa, more particularly the flash point is less than 90 °C and the vapour pressure is greater than 1 Pa, even more particularly the flash point is less than or equal to 60 °C and the vapour pressure is greater than 5 Pa, and even more particularly still the flash point is less than 60 °C and the vapour pressure is greater than 100 Pa.

The volatile oil(s) may be selected from volatile hydrocarbon oils such as:
hydrocarbon oils having from 8 to 16 carbon atoms, and particularly:
a) branched C8-C16 alkanes such as isoalkanes (also known as isoparaffins) such as C8-C9 Isoparaffin, C13-C16 Isoparaffin, isododecane, isodecane, isohexadecane, and for example oils sold under the trade names Isopar or Permethyl, alone or as mixtures, in particular isododecane (also known as 2,2,4,4,6-pentamethylheptane), for example sold by Ineos, more particularly isododecane;
b) linear C6-C16 alkanes, alone or as mixtures, for example such as hexane, decane, undecane, tridecane, or n-dodecane (C12) and n-tetradecane (C14) sold by Sasol under the respective references Parafol 12-97 and Parafol 14-97, the undecane-tridecane mixture, the mixtures of n-undecane (C11) and n-tridecane (C13) obtained in Examples 1 and 2 of patent application WO 2008/155059 by the company Cognis, and mixtures thereof, and also mixtures of n-undecane (C11) and n-tridecane (C13) Cetiol Ultimate® from the company BASF;
c) volatile C5-C12 cyclic, non-aromatic alkanes;
short-chain esters having 3 to 8 carbon atoms in total, such as methyl acetate, ethyl acetate, propyl acetate, n-butyl acetate or isobutyl acetate, for example sold by Solvay, Dow or Oxea;
volatile carbonate hydrocarbon oils of structure R′1-O—C(O)—O—R′2 in which R′1 and R′2, which may be identical or different, independently denote a linear, branched or cyclic C4-C8 alkyl group, in particular a linear C4-C8 alkyl group. It may be preferable for [Chem 1]R1 and R2 to be identical. In particular, R′1 and R′2 denote a linear butyl alkyl radical or a pentyl group.

Advantageously, the ether oil is selected from dibutyl carbonate or dipentyl carbonate;
volatile ether oils of formula R1-O—R2 in which R1 and R2, which may be identical or different, independently denote a linear, branched or cyclic C4-C8 alkyl group, in particular a linear or branched C4-C8 alkyl group. It is preferable for R1 and R2 to be identical. Linear alkyl groups that may be mentioned include a butyl group and a pentyl group. Branched alkyl groups that may be mentioned include a 1-methylpropyl group, a 2-methylpropyl group, a t-butyl group and a 1,1-dimethylpropyl group.

In particular, the volatile hydrocarbon oil(s) are selected from C8-C16 alkanes, in particular linear C8-C16 alkanes, and more particularly are selected from C9-C12 alkanes, even more particularly are selected from a mixture of C9-C12 alkanes such as Vegelight Silk® sold by BioSynthIs.

The volatile oil(s) may be selected from volatile silicone oils such as:
silicone oils comprising in particular from 2 to 7 silicon atoms, these silicone oils optionally including alkyl or alkoxy groups having from 1 to 10 carbon atoms. As volatile silicone oils that may be used in the invention, mention may particularly be made of dimethicones with viscosities of 5 and 6 cSt, cyclopentadimethylsiloxane, dodecamethylpentasiloxane, cyclohexadimethylsiloxane, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof. Mention may particularly be made of dodecamethylpentasiloxane, such as the reference DM-Fluid-2cs sold by Shin-Etsu, or cyclohexadimethylsiloxane, such as the reference Xiameter PMX-0246 Cyclohexasiloxane sold by Dow Chemical.

### Non-Volatile Oils

“Non-volatile oil” means an oil, the vapour pressure of which at 25 °C and atmospheric pressure is non-zero and is less than 2.66 Pa and more particularly less than 0.13 Pa. By way of example, the vapour pressure may be measured according to the static method or via the effusion method by isothermal thermogravimetry, depending on the vapour pressure of the oil (standard OCDE 104).

The non-volatile oil(s) may be of natural or synthetic origin, in particular natural.

Among the non-volatile oils, mention may be made of:
non-volatile fluoro oils, which may particularly be selected from fluorinated polyethers, and also from the fluorosilicone oils and the fluoro silicones as described in EP-A-847752;
non-volatile silicone oils, which may particularly be selected from the non-volatile silicones having the following INCI names: dimethicone, dimethiconol, trimethyl pentaphenyl trisiloxane, tetramethyl tetraphenyl trisiloxane, diphenyl dimethicone, trimethylsiloxyphenyl dimethicone, phenyl trimethicone, diphenylsiloxyphenyl trimethicone; and also mixtures thereof.

These products are particularly sold under the names PH-1555 HRI Cosmetic Fluid (trimethyl pentaphenyl trisiloxane), Dow Corning 556 Cosmetic Grade Fluid (phenyl trimethicone) by Dow Corning; diphenyl dimethicones such as the products KF-54, KF54HV, KF-50-300CS, KF-53 d and KF-50-100CS or Diphenylsiloxy Phenyl Trimethicone KF56 A sold by Shin-Etsu; the products Belsil PDM 1000 and Belsil PDM 20 sold by Wacker Chemie (trimethylsiloxy phenyl dimethicone), alone or as mixtures;
apolar non-volatile hydrocarbon oils, which may particularly be selected from linear or branched compounds of mineral or synthetic origin, for instance: i) squalane such as the reference Neossance Squalane sold by Amyris, isoeicosane, ii) mixtures of linear, saturated hydrocarbons, particularly C14-C30 and more particularly C15-C28 hydrocarbons, such as mixtures with, for example, the following INCI names: (C15-C19)Alkane, (C18-C21)Alkane, (C21-C28)Alkane, for instance the products Gemseal 40, Gemseal 60 and Gemseal 120 sold by Total, Emogreen L19 sold by SEPPIC, Emogreen L15 sold by SEPPIC, iii) hydrogenated or non-hydrogenated polybutenes, for instance the products of the Indopol range sold by Ineos Oligomers, products having the INCI name Hydrogenated Polyisobutene; iv) hydrogenated or non-hydrogenated polyisobutenes, in particular hydrogenated, for instance the non-volatile compounds of the Parleam® range sold by the company Nippon Oil Fats, v) hydrogenated or non-hydrogenated polydecenes, for instance the non-volatile compounds of the Puresyn® range sold by the company ExxonMobil), vi) decene/butene copolymers, butene/isobutene copolymers, and vii) mixtures thereof:
non-volatile polar hydrocarbon oils, which may be selected from:
i) saturated, unsaturated, linear or branched C10-C26 fatty alcohols, which are liquid at room temperature (25 °C), in particular monoalcohols. In particular, the C10-C26 alcohols are fatty alcohols, which are in particular branched when they comprise at least 16 carbon atoms; in particular, the fatty alcohol comprises from 10 to 24 carbon atoms, and more particularly from 12 to 22 carbon atoms, particularly such as lauryl alcohol, isostearyl alcohol, oleyl alcohol, 2-butyloctanol, 2-undecylpentadecanol, 2-hexyldecyl alcohol, isocetyl alcohol, octyldodecanol and mixtures thereof;
ii) triglycerides consisting of fatty acid esters of glycerol, in particular the fatty acids of which may have chain lengths ranging from C4 to C36, and particularly from C18 to C36, it being possible for these oils to be linear or branched, saturated or unsaturated; by way of example, mention may particularly be made of heptanoic or octanoic triglycerides, caprylic/capric acid triglycerides, plant oils such as wheat germ oil, sunflower oil, grapeseed oil, sesame seed oil, corn oil, apricot kernel oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil, musk rose oil, groundnut oil, coconut oil, argan oil, passionflower oil, kaya oil; the liquid fraction of shea butter, and the liquid fraction of cocoa butter; and also mixtures thereof;
iii) linear aliphatic hydrocarbon esters of formula R—C(O)—OR′ in which R—C(O)— represents a carboxylic acid residue including from 2 to 40 carbon atoms and R′ represents a hydrocarbon chain containing from 1 to 40 carbon atoms, aliphatic hydrocarbon esters of alkylene glycol, in particular ethylene glycol or propylene glycol, the total number of carbon atoms advantageously being at least 10. As examples of such esters, mention may for example be made of isoamyl laurate, cetostearyl octanoate, isopropyl stearate or isostearate, ethyl palmitate, 2-ethylhexyl palmitate, isostearyl isostearate, octyl stearate, isostearyl heptanoate, octanoates, decanoates or ricinoleates of alcohols or of polyalcohols, such as propylene glycol dioctanoate, cetyl octanoate, cocoyl caprylate/caprate, tridecyl octanoate, 2-ethylhexyl palmitate, alkyl benzoate, polyethylene glycol diheptanoate, propylene glycol bis(2-ethylhexanoate) and mixtures thereof, hexyl laurate, neopentanoic acid esters, such as isodecyl neopentanoate, isotridecyl neopentanoate, isostearyl neopentanoate or 2-octyldodecyl neopentanoate, isononanoic acid esters, such as isononyl isononanoate, isotridecyl isononanoate or octyl isononanoate, oleyl erucate, isopropyl lauroyl sarcosinate, diisopropyl sebacate, isocetyl stearate, isodecyl neopentanoate, isostearyl behenate or myristyl myristate; and mixtures thereof;
iv) hydroxylated esters such as polyglyceryl-2 triisostearate;
v) aromatic esters such as tridecyl trimellitate, C12-C15 alcohol benzoate, the 2-phenylethyl ester of benzoic acid, and butyloctyl salicylate;
vi) linear fatty acid esters having a total carbon number ranging from 35 to 70, for instance pentaerythrityl tetrapelargonate;
vii) esters of C24-C28 branched fatty acids or fatty alcohols such as triisoarachidyl citrate, pentaerythrityl tetraisononanoate, glyceryl triisostearate, glyceryl tris(2-decyltetradecanoate), pentaerythrityl tetraisostearate, polyglyceryl-2 tetraisostearate or pentaerythrityl tetrakis(2-decyltetradecanoate);
viii) the polyesters obtained by condensation of dimer and/or trimer of unsaturated fatty acid and of diol, such as those with the INCI name Dilinoleic Acid/Butanediol Copolymer or Dilinoleic Acid/Propanediol Copolymer; the polyesters obtained by condensation of fatty acid dimer and of diol dimer, such as dimer dilinoleyl dimer dilinoleate;
ix) synthetic ethers of formula R1-O—R2 in which R1 and R2, which may be identical or different, independently denote a linear, branched or cyclic C6-C24 alkyl group, in particular a C6-C18 alkyl group, and more particularly a C8-C12 alkyl group. It may be preferable for R1 and R2 to be identical. Linear alkyl groups that may be mentioned include a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a behenyl group, a docosyl group, a tricosyl group and a tetracosyl group. Branched alkyl groups that may be mentioned include a 1,1-dimethylpropyl group, a 3-methylhexyl group, a 5-methylhexyl group, an ethylhexyl group, a 2-ethylhexyl group, a 5-methyloctyl group, a 1-ethylhexyl group, a 1-butylpentyl group, a 2-butyloctyl group, an isotridecyl group, a 2-pentylnonyl group, a 2-hexyldecyl group, an isostearyl group, a 2-heptylundecyl group, a 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyl group and a 2-(1,3,3-trimethylbutyl)-5,7,7-trimethyloctyl group.

As cyclic alkyl groups, mention may be made of a cyclohexyl group, a 3-methylcyclohexyl group and a 3,3,5-trimethylcyclohexyl group, dilauryl ether, diisostearyl ether, dioctyl ether, nonylphenyl ether, dodecyl dimethylbutyl ether, cetyl dimethylbutyl ether, cetyl isobutyl ether and mixtures thereof. Among the non-volatile ether oils, mention may be made of dicaprylyl ether, such as the reference Cetiol OE sold by BASF;
x) carbonates of formula R8-O—C(O)—O—R9, with R8 and R9, which may be identical or different, representing a linear or branched C4 to C12 and in particular C6 to C10 alkyl chain; the carbonate oils may be dicaprylyl carbonate (or dioctyl carbonate), sold under the name Cetiol CC® by the company BASF, bis(2-ethylhexyl) carbonate, sold under the name Tegosoft DEC® by the company Evonik, dipropylheptyl carbonate (Cetiol 4 All from BASF), dibutyl carbonate, dineopentyl carbonate, dipentyl carbonate, dineoheptyl carbonate, diheptyl carbonate, diisononyl carbonate or dinonyl carbonate, and more particularly dioctyl carbonate;
xi) vinylpyrrolidone copolymers such as vinylpyrrolidone/1-hexadecene copolymer (INCI name);
xii) higher C6-C26 fatty acids which are liquid at room temperature (25 °C), such as oleic acid, linoleic acid, linolenic acid or isostearic acid; and
xiii) mixtures thereof.

According to a particular embodiment, the composition comprises at least one oil selected from volatile C8-C16 alkane hydrocarbon oils, non-volatile silicone oils, apolar non-volatile hydrocarbon oils with the exception of liquid paraffins, polar non-volatile hydrocarbon oils as defined previously, and mixtures thereof more particularly selected from polar non-volatile hydrocarbon oils and mixtures thereof.

According to a particular embodiment, the composition comprises at least one oil selected from polar non-volatile hydrocarbon oils; more particularly, the composition according to the invention comprises at least one oil selected from polar non-volatile hydrocarbon oils and does not comprise any apolar non-volatile hydrocarbon oils; even more particularly, the composition according to the invention comprises at least one oil selected from polar non-volatile hydrocarbon oils and does not comprise any apolar non-volatile hydrocarbon oils, non-volatile fluoro oils, non-volatile silicone oils or volatile oils.

According to a particular embodiment, the polar non-volatile hydrocarbon oils are selected from:
i) saturated, unsaturated, linear or branched C10-C26 fatty alcohols, which are liquid at room temperature (25 °C), in particular monoalcohols. In particular, the C10-C26 alcohols are fatty alcohols, which are in particular branched when they comprise at least 16 carbon atoms; more particularly, the fatty alcohol comprises from 10 to 24 carbon atoms, and even more particularly from 12 to 22 carbon atoms, particularly such as lauryl alcohol, isostearyl alcohol, oleyl alcohol, 2-butyloctanol, 2-undecylpentadecanol, 2-hexyldecyl alcohol, isocetyl alcohol, octyldodecanol and mixtures thereof;
ii) triglycerides consisting of fatty acid esters of glycerol, in particular the fatty acids of which may have chain lengths ranging from C4 to C36, and particularly from C18 to C36, it being possible for these oils to be linear or branched, saturated or unsaturated; by way of example, mention may particularly be made of heptanoic or octanoic triglycerides, caprylic/capric acid triglycerides, plant oils such as wheat germ oil, sunflower oil, grapeseed oil, sesame seed oil, corn oil, apricot kernel oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil, musk rose oil, groundnut oil, coconut oil, argan oil, passionflower oil, kaya oil; the liquid fraction of shea butter, and the liquid fraction of cocoa butter; and also mixtures thereof;
iii) linear aliphatic hydrocarbon esters of formula R—C(O)—OR′ in which R—C(O)— represents a carboxylic acid residue including from 2 to 40 carbon atoms and R′ represents a hydrocarbon chain containing from 1 to 40 carbon atoms, aliphatic hydrocarbon esters of alkylene glycol, in particular ethylene glycol or propylene glycol, the total number of carbon atoms advantageously being at least 10. As examples of such esters, mention may for example be made of isoamyl laurate, cetostearyl octanoate, isopropyl stearate or isostearate, ethyl palmitate, 2-ethylhexyl palmitate, isostearyl isostearate, octyl stearate, isostearyl heptanoate, octanoates, decanoates or ricinoleates of alcohols or of polyalcohols, such as propylene glycol dioctanoate, cetyl octanoate, cocoyl caprylate/caprate, tridecyl octanoate, 2-ethylhexyl palmitate, alkyl benzoate, polyethylene glycol diheptanoate, propylene glycol bis(2-ethylhexanoate) and mixtures thereof, hexyl laurate, neopentanoic acid esters, such as isodecyl neopentanoate, isotridecyl neopentanoate, isostearyl neopentanoate or 2-octyldodecyl neopentanoate, isononanoic acid esters, such as isononyl isononanoate, isotridecyl isononanoate or octyl isononanoate, oleyl erucate, isopropyl lauroyl sarcosinate, diisopropyl sebacate, isocetyl stearate, isodecyl neopentanoate, isostearyl behenate or myristyl myristate; and mixtures thereof;
ix) synthetic ethers of formula R1-O—R2 in which R1 and R2, which may be identical or different, independently denote a linear, branched or cyclic C6-C24 alkyl group, in particular a C6-C18 alkyl group, and more particularly a C8-C12 alkyl group. It may be preferable for R1 and R2 to be identical. Linear alkyl groups that may be mentioned include a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a behenyl group, a docosyl group, a tricosyl group and a tetracosyl group. Branched alkyl groups that may be mentioned include a 1,1-dimethylpropyl group, a 3-methylhexyl group, a 5-methylhexyl group, an ethylhexyl group, a 2-ethylhexyl group, a 5-methyloctyl group, a 1-ethylhexyl group, a 1-butylpentyl group, a 2-butyloctyl group, an isotridecyl group, a 2-pentylnonyl group, a 2-hexyldecyl group, an isostearyl group, a 2-heptylundecyl group, a 2-octyldodecyl group, a 1,3-dimethylbutyl group, a 1-(1-methylethyl)-2-methylpropyl group, a 1,1,3,3-tetramethylbutyl group, a 3,5,5-trimethylhexyl group, a 1-(2-methylpropyl)-3-methylbutyl group, a 3,7-dimethyloctyl group and a 2-(1,3,3-trimethylbutyl)-5,7,7-trimethyloctyl group. Cyclic alkyl groups that may be mentioned include a cyclohexyl group, a 3-methylcyclohexyl group and a 3,3,5-trimethylcyclohexyl group, dilauryl ether, diisostearyl ether, dioctyl ether, nonylphenyl ether, dodecyl dimethylbutyl ether, cetyl dimethylbutyl ether and mixtures thereof. Among the non-volatile ether oils, mention may be made of dicaprylyl ether, such as the reference Cetiol OE sold by BASF;
x) carbonates of formula R8-O—C(O)—O—R9, with R8 and R9, which may be identical or different, representing a linear or branched C4 to C12 and in particular C6 to C10 alkyl chain; the carbonate oils may be dicaprylyl carbonate (or dioctyl carbonate), sold under the name Cetiol CC® by the company BASF, bis(2-ethylhexyl) carbonate, sold under the name Tegosoft DEC® by the company Evonik, dipropylheptyl carbonate (Cetiol 4 All from BASF), dibutyl carbonate, dineopentyl carbonate, dipentyl carbonate, dineoheptyl carbonate, diheptyl carbonate, diisononyl carbonate or dinonyl carbonate, and more particularly dioctyl carbonate.

More particularly, the polar non-volatile hydrocarbon oils are selected from:
ii) triglycerides consisting of fatty acid esters of glycerol, in particular the fatty acids of which may have chain lengths ranging from C4 to C36, and particularly from C18 to C36, it being possible for these oils to be linear or branched, saturated or unsaturated; by way of example, mention may particularly be made of heptanoic or octanoic triglycerides, caprylic/capric acid triglycerides, plant oils such as wheat germ oil, sunflower oil, grapeseed oil, sesame seed oil, corn oil, apricot kernel oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passionflower oil, musk rose oil, groundnut oil, coconut oil, argan oil, passionflower oil, kaya oil; the liquid fraction of shea butter, and the liquid fraction of cocoa butter; and also mixtures thereof.

According to a particular embodiment, the composition comprises at least one oil selected from the group consisting of:
polar non-volatile hydrocarbon oils of the triglyceride type consisting of fatty acid esters of glycerol, in particular the fatty acids of which may have chain lengths ranging from C4 to C36, and particularly from C18 to C36, it being possible for these oils to be linear or branched, saturated or unsaturated, selected from soybean oil, jojoba seed oil, shea butter olein, capric and caprylic acid triglycerides, and mixtures thereof,
   volatile C8-C16 alkane oils, such as C9-C12 alkanes and isoparaffin,
apolar non-volatile oils such as squalane,
polar non-volatile hydrocarbon oils of the synthetic ether type of formula R1-O—R2, in which R1 and R2, which may be identical or different, independently denote a linear, branched or cyclic C6-C24 alkyl group, in particular a C6-C18 alkyl group, and more particularly a C8-C12 alkyl group, such as dicaprylyl ether,
non-volatile silicone oils such as dimethicone,
polar non-volatile hydrocarbon oils of the saturated, unsaturated, linear or branched C10-C26 fatty alcohol type, which are liquid at room temperature (25 °C), such as octyldodecanol,
polar non-volatile hydrocarbon oils such as the carbonates of formula R8-O—C(O)—O—R9, in which R8 and R9, which may be identical or different, represent a linear or branched C4 to C12, in particular C6 to C10, alkyl chain, such as dicaprylyl carbonate, and
mixtures thereof.

According to a particular embodiment, the oil is selected from the group consisting of octyldodecanol, soybean oil, shea butter olein, dicaprylyl carbonate, dimethicone, jojoba oil, isoparaffin, isononyl isonanoate, caprylic/capric acid triglycerides, C9-C12 alkanes, squalane, dicaprylyl ether, and mixtures thereof.

According to a particular embodiment, the oil is selected from the group consisting of soybean oil, shea butter olein, jojoba oil, isononyl isonanoate, caprylic/capric acid triglycerides, and mixtures thereof.

According to a particular embodiment, the oil is an oil which is different from a liquid paraffin.

According to a particular embodiment, the composition is free of liquid paraffins, i.e. the composition comprises 0% liquid paraffins.

The oils according to the invention may advantageously be present in a composition according to the invention in a content customary for a cosmetic composition, in particular in a customary content allowing them to play their cosmetic role in a composition of the invention, in particular in a cosmetic composition according to the invention, more particularly in a customary content allowing them to play their cosmetic role when they are the only ones to play this role in a composition according to the invention.

The oil may play various cosmetic roles, such as that of a consistency factor, sustaining an emulsion under cold conditions, or obtaining the smooth appearance of the composition, particularly in the case of an emulsion. It may also contribute towards facilitating the spreading and gliding of the composition over the skin, and also its penetration. Finally, the oil may act on the skin through its occlusive effect, its lubricating effect (to the touch) or its emollient/hydrating effect.

A composition according to the invention may comprise a total oil content ranging from 1% to 80% by weight relative to the total weight of the composition, in particular from 2% to 60% by weight relative to the total weight of the composition, more particularly from 5% to 40% by weight, even more particularly from 10% to 30% by weight relative to the total weight of the composition.

“Total oil content” means the sum of the contents of each of the previously mentioned oils present in the composition, or, when only one of these oils is present in the composition, it means the content of this oil.

### Aromatic Alcohols

A composition according to the invention may also comprise at least one aromatic alcohol of formula (III), a salt thereof, particularly a salt of an organic or mineral base thereof, an optical isomer thereof, a geometrical isomer thereof or a solvate thereof, such as hydrates:

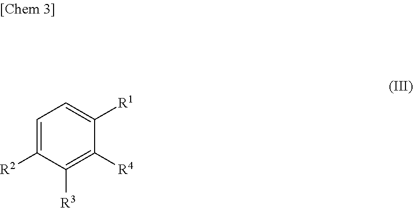

in which
R¹ represents a group selected from the group consisting of:
a) linear or branched hydroxy(C₁-C₄)alkyl, in particular a hydroxy(C₁-C₂)alkyl group,
b) a group selected from —OR⁵, —C(O)R⁶, —C(O)OR⁷, and —(CH₂)ₙ—C(H)(R⁸)—C(O)R⁹, with:
R⁵ representing a linear or branched (C₃-C₄)alkyl group optionally substituted with one or more hydroxyl (OH) groups,
R⁶ representing a hydrogen atom or a linear or branched (C₁-C₄)alkyl group, a phenyl or a benzyl, optionally substituted with one or more hydroxyl group(s),
R⁷ representing a linear or branched (C₁-C₄)alkyl group, a phenyl or a benzyl, optionally substituted with one or more hydroxyl group(s),
R⁸ representing a hydrogen atom or linear or branched (C₁-C₄)alkyl group such as methyl or ethyl; in particular, R⁸ representing a hydrogen atom;
R⁹ representing a hydrogen atom or a linear or branched, in particular linear, (C₁-C₁₂)alkyl group, optionally substituted with one or more hydroxyl group(s), or else a linear or branched, in particular linear, (C₂-C₁₂)alkenyl group, optionally substituted with one or more hydroxyl group(s),
n is 0, 1 or 2; in particular, n is 1
R² represents a hydrogen atom, a halogen atom, in particular a chlorine atom, or a hydroxyl group; and
R³ represents a hydrogen atom or a group selected from hydroxyl and linear or branched (C₁-C₆)alkoxy, in particular (C₁-C₄)alkoxy such as methoxy —OCH₃, ethoxy —OC₂H₅; in particular, R³ represents a hydrogen atom or an ethoxy group; and
R⁴ is a hydrogen atom or a hydroxyl group;
it being understood that at least one of the groups R¹, R², R³ or R⁴ bears or represents a hydroxyl group.

More particularly, the aromatic alcohol(s) of formula (I) of the invention are such that:
when R¹ represents a group —C(O)OR⁷, then R² is an —OH group;
when R¹ represents a group —C(O)R⁶, then at least one of R², R³ and R⁴ is an —OH group;
when R¹ represents a group —(CH₂)ₙ—C(H)(R⁸)—C(O)R⁹, then R² is an —OH group.

In particular, the aromatic alcohol may have the following formula (IV):

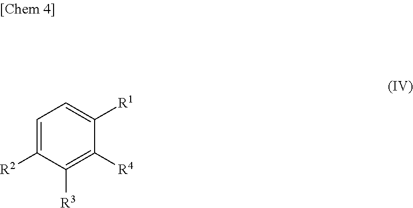

in which
R¹ is selected from the group consisting of
i) a linear or branched hydroxy(C₁-C₄)alkyl group, in particular a hydroxy(C₁-C₂)alkyl group such as hydroxymethyl or hydroxyethyl,
ii) an —OR⁵ group, with R⁵ representing a (C₃-C₄)hydroxyalkyl group; —OR⁵ particularly representing —O—CH₂—CH(OH)—CH₂OH,
iii) a —C(O)R⁶ group, with R⁶ representing a linear or branched (C₁-C₄)alkyl group; —C(O)R⁶ particularly representing —C(O)—CH₃,
iv) a —C(O)OR⁷ group, with R⁷ representing a linear or branched (C₁-C₄)alkyl group; R⁷ particularly representing a methyl, ethyl, propyl, isopropyl, butyl, isobutyl or benzyl group, and
v) a —CH₂—CH₂—C(O)R⁹ group, with R⁹ representing a linear or branched (C₁-C₆)alkyl group; in particular, —CH₂—CH₂—C(O)R⁹ representing a —CH₂—CH₂—C(O)CH₃ group;
R² is selected from the group consisting of a hydrogen atom, a halogen atom, in particular a chlorine atom, and a hydroxyl group;
R³ is a hydrogen atom, a methoxy or ethoxy group; and
R⁴ is a hydrogen atom or a hydroxyl group;
it being understood that at least one of the groups R¹, R² or R⁴ bears or represents a hydroxyl group.

Such compounds particularly act as preserving agents, in particular in cosmetic compositions. Some also act as fragrances, particularly in cosmetic compositions.

The term “organic or mineral base salt” means salts of bases or alkaline agents as defined below. As examples of base salts, mention may be made of alkali metal hydroxides such as sodium, potassium and lithium hydroxides; alkaline-earth metal hydroxides such as calcium and magnesium hydroxides; hydroxides of other metals, such as aluminium and zinc hydroxides; aqueous ammonia and organic amines such as unsubstituted or hydroxy-substituted mono-, di- or tri-alkylamines; dicyclohexylamines; tributylamines; pyridine; N-methyl-N-ethylamine; diethylamine; triethylamine; mono-, bis- or tris-(2-hydroxyalkylamines) such as mono-, bis- or tris-(2-hydroxyethyl)amine, 2-hydroxy-tert-butylamine, tris(hydroxymethyl)methylamine; N,N-dialkyl-N-(hydroxyalkyl)amines, such as N,N-dimethyl-N-(2-hydroxyethyl)amine; N-methyl-D-glucamine; and amino acids such as arginine and lysine.

For the purposes of the present invention, “alkyl group” or “alkyl radical” means a saturated, linear or branched, substituted or unsubstituted monovalent hydrocarbon radical, in particular methyl, ethyl, propyl, isopropyl, butyl or tert-butyl radicals.

“Hydroxyalkyl group” means a saturated, linear or branched hydrocarbon group comprising at least one —OH group. A hydroxy(C₁-C₄)alkyl group is a saturated, linear or branched C₁-C₄ hydrocarbon radical comprising at least one —OH group, in particular comprising a single —OH group. A hydroxy(C₁-C₂)alkyl group is a saturated, linear or branched C₁-C₂ hydrocarbon radical comprising at least one —OH group, in particular comprising a single —OH group.

According to a particular embodiment, the hydroxy(C₁-C₄)alkyl group is selected from the group consisting of hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl. In particular, the hydroxyl(C₁-C₄)alkyl group is hydroxymethyl or 2-hydroxyethyl.

“Halogen atom” means one of the chemical elements of Group 17 of the Periodic Table of the Elements, namely fluorine, chlorine, bromine or iodine. In particular, the halogen atom is a chlorine atom.

According to a particular embodiment, R¹ is selected from the group consisting of (C₁-C₂)hydroxyalkyl, 2-hydroxyethyl, hydroxymethyl, a —O—CH₂—CH(OH)—CH₂OH group, a —CH₂—CH₂—C(O)—CH₃ group, and a —C(O)—OCH₃ group,
R² is selected from the group consisting of a hydrogen atom, a halogen atom, in particular a chlorine atom, and a hydroxyl group,
R³ is selected from the group consisting of —O—C₂—H₅, and
R⁴ is a hydrogen atom.

According to one embodiment, R¹ represents a hydroxy(C₁-C₄)alkyl group, more particularly a hydroxy(C₁-C₂)alkyl group, R² represents a hydrogen atom, R³ represents a hydrogen atom and R⁴ represents a hydrogen atom.

According to a particular embodiment, R¹ represents 2-hydroxyethyl, R² represents a hydrogen atom, R³ represents a hydrogen atom and R⁴ represents a hydrogen atom.

According to a particular embodiment, R¹ represents a hydroxymethyl, R² represents a hydrogen atom, R³ represents a hydrogen atom and R⁴ represents a hydrogen atom.

According to a particular embodiment, R¹ represents an —OR⁵ group with R⁵ representing a linear or branched (C₃-C₄)alkyl group substituted with one or more hydroxyl group(s), R² represents a halogen atom, R³ represents a hydrogen atom and R⁴ represents a hydrogen atom. More particularly, R¹ represents an —OR⁵ group with R⁵ representing a linear (C₃-C₄)alkyl group substituted with two hydroxyl groups, R² represents a halogen atom, R³ represents a hydrogen atom and R⁴ represents a hydrogen atom.

According to a particular embodiment, R¹ represents an —O—CH₂—CH(OH)—CH₂OH group, R² represents a chlorine atom, R³ represents a hydrogen atom and R⁴ represents a hydrogen atom.

According to a particular embodiment, R¹ represents a —(CH₂)ₙ—C(H(R)⁸—C(O)R⁹ group, with R⁸ representing a hydrogen atom or a methyl or ethyl group, and R⁹ representing a linear (C₁-C₁₂)alkyl group optionally substituted with a hydroxyl group or a (C₂-C₁₂)alkenyl group optionally substituted with a hydroxyl group, and n is as defined previously; in particular, n is 1, R² represents a hydroxyl group, R³ represents an —OCH₃ or —OC₂H₅ group and R⁴ represents a hydrogen atom. More particularly, R¹ represents a —CH₂—CH₂—C(O)R⁹ group, R⁹ representing a linear (C₁-C₁₂)alkyl group, in particular a linear (C₁-C₆)alkyl such as methyl, R² represents a hydroxyl group, R³ represents an —OC₂H₅ group and R⁴ represents a hydrogen atom.

According to a particular embodiment, R¹ represents a —C(O)OR⁷ group, with R⁷ representing a linear or branched (C₁-C₄)alkyl group, a phenyl or a benzyl, optionally substituted with one or more hydroxyl group(s), R² represents a hydroxyl group, R³ represents a hydrogen atom and R⁴ represents a hydrogen atom.

According to a particular embodiment, R¹ represents a —C(O)—OCH₃ group, R² represents a hydroxyl group, R³ represents a hydrogen atom and R⁴ represents a hydrogen atom.

According to a particular embodiment, the aromatic alcohol of formula (III) or (IV) is selected from the group consisting of phenylethyl alcohol; benzyl alcohol; chlorphenesin (also known as 3-(4-chlorophenoxy)-1,2-propanediol); zingerone; ethylzingerone (also known as 4-(3-ethoxy-4-hydroxyphenyl)butan-2-one); vanillin; parabens, in particular (C₁-C₆)alkylparabens or arylparabens, in particular methylparaben, ethylparaben, propylparaben, isopropylparaben, butylparaben, isobutylparaben or benzylparaben; salts thereof and mixtures thereof.

According to a particular embodiment, the aromatic alcohol of formula (III) or (IV) is selected from the group consisting of phenylethyl alcohol; benzyl alcohol; chlorphenesin (also known as 3-(4-chlorophenoxy)-1,2-propanediol); zingerone; ethylzingerone (also known as 4-(3-ethoxy-4-hydroxyphenyl)butan-2-one); parabens, in particular methylparaben; salts thereof and mixtures thereof.

The composition according to the invention comprises in particular a total content of aromatic alcohol(s) of formula (III) or (IV) ranging from 0.01% to 3% by weight relative to the total weight of the composition, in particular from 0.05% to 1.5% by weight, and more particularly from 0.1% to 1.0% by weight, relative to the total weight of the composition.

“Total content of aromatic alcohol(s) of formula (III) or (IV)” means the sum of the contents of each of the aromatic alcohol(s) of formula (III) or (IV) present in the composition, or, when only one aromatic alcohol of formula (III) or (IV) is present in the composition, means the content of this aromatic alcohol of formula (III) or (IV).

### Organic Fillers

The composition according to the invention may also comprise one or more organic filler(s).

For the purposes of the present invention, “organic filler” refers to colourless or white organic, natural or synthetic solid particles of any form, which are in a form that is insoluble and dispersed in the medium of the composition.

The composition according to the invention may also comprise at least one organic filler selected from an unmodified starch, an N-acylamino acid, salts thereof and mixtures thereof.

### Unmodified Starches

The composition according to the present invention may comprise one or more unmodified starch(es).

For the purposes of the present invention, “unmodified starch” means native starch, or else starch which has not been chemically or physically modified, particularly by one or more of the following reactions: pregelatinization, oxidation, crosslinking, esterification, etherification, amidation, heat treatment.

The unmodified starch molecules that may be used in the present invention may originate from any plant source of starch, particularly cereals and tubers; more particularly, they may be starches from corn, rice, cassava, barley, potato, wheat, sorghum, pea or oat.

According to a particular embodiment, the unmodified starch is selected from corn starches, rice starches, potato starches and mixtures thereof; in particular, the unmodified starch is selected from corn or potato starches.

According to a particular embodiment, the composition according to the invention is totally free of tapioca starch; in particular, the composition according to the invention is totally free of unmodified tapioca starch.

According to a particular embodiment, the unmodified starch used in the composition of the present invention is a corn starch such as that sold under the name Beauty-by-Roquette ST005 by the company Roquette.

The starch(es) may be present in the composition according to the invention in a content ranging from 0.1% to 10% by weight, in particular from 0.5% to 5% by weight, more particularly from 1% to 2.5% by weight, such as 1%, 1.5% or 2% by weight, relative to the total weight of the composition.

### N-Acylamino Acids and Salts Thereof

A composition according to the present invention may comprise one or more N-acylamino acids, salts thereof and mixtures thereof.

“Salt” of an N-acylamino acid according to the invention means a salt formed by an inorganic or organic acid or an inorganic or organic base.

As examples of acid salts, mention may be made of the sulfate, citrate, acetate, oxalate, chloride, bromide, iodide, nitrate, bisulfate, phosphate, isonicotinate, lactate, salicylate, tartrate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and aspartate salts.

As examples of base salts, mention may be made of hydroxides of alkali metals such as sodium, potassium and lithium; hydroxides of alkaline-earth metals such as calcium and magnesium; hydroxides of other metals such as aluminium and zinc; aqueous ammonia and organic amines such as unsubstituted or hydroxy-substituted mono-, di- or trialkylamines; dicyclohexylamines; tributylamines; pyridine; N-methyl-N-ethylamine; diethylamine; triethylamine; mono-, bis- or tris(2-hydroxyalkylamines) such as mono-, bis- or tris(2-hydroxyethyl)amine, 2-hydroxy-tert-butylamine or tris(hydroxymethyl)methylamine, N,N-di-alkyl-N-(hydroxyalkyl)amines, such as N,N-dimethyl-N-(2-hydroxyethyl)amine; N-methyl-D-glucamine; and amino acids such as arginine and lysine.

N-Acylamino acids that are suitable as organic fillers according to the invention comprise at least one acyl group having from 8 to 22 carbon atoms, in particular a 2-ethylhexanoyl, caproyl, lauroyl, myristoyl, palmitoyl, stearoyl or cocoyl group, in particular lauroyl.

The amino acid may be, for example, lysine, glutamic acid or alanine, preferably lysine.

The amino acid may be of D or L configuration, in particular L.

According to a particular embodiment of the invention, the C8-C22 N-acylamino acids are selected from lauroyl lysine, salts thereof and mixtures thereof, in particular N-lauroyl-L-lysine, salts thereof and mixtures thereof.

N-Lauroyl-L-lysine is particularly sold under the name Amihope LL® by the company Ajinomoto.

The N-acylamino acid(s) and the salts thereof may be present in the composition according to the invention in a content ranging from 0.1% to 15% by weight, in particular from 1% to 10% by weight, more particularly from 2% to 4% by weight, such as 3% by weight, relative to the total weight of the composition.

The organic filler(s) may be present in the composition according to the invention in a total content ranging from 0.1% to 15% by weight, in particular from 0.5% to 10% by weight, more particularly from 1% to 4% by weight, relative to the total weight of the composition.

A composition according to the invention may comprise one or more endolysin(s) according to the invention and one or more organic filler(s) in an endolysin(s)/organic filler(s) mass ratio of between 0.0001 and 0.04, in particular between 0.0002 and 0.004 and more particularly between 0.0006 and 0.002.

According to a particular embodiment, a composition according to the invention comprises an amount of less than 2% by weight, relative to the total weight of the composition, of fatty acid(s) that are solid at room temperature (25 °C); more particularly, a composition according to the invention comprises an amount of less than 1% by weight, relative to the total weight of the composition, of fatty acid(s) that are solid at room temperature (25 °C), or even is free of (0% by weight relative to the total weight of the composition) fatty acid(s) that are solid at room temperature (25 °C), and in particular does not comprise any stearic acid.

According to a particular embodiment, a composition according to the invention comprises an amount of less than 0.5% by weight, relative to the total weight of the composition, of benzoic acid and/or salts thereof, particularly alkali metal or alkaline-earth metal benzoates such as sodium benzoate, of sorbic acid and/or salts thereof, particularly alkali metal or alkaline-earth metal sorbates, including potassium sorbate, more particularly less than 0.1% by weight, or even is free of (0% by weight relative to the total weight of the composition) benzoic acid and/or salts thereof, including sodium benzoate, and sorbic acid and/or salts thereof, including potassium sorbate.

According to a particular embodiment, a composition according to the invention comprises an amount of less than 0.1% in total amount by weight, relative to the total weight of the composition, of carrageenan, gellan gum, scleroglucan gum, gum arabic, pectin, xanthan gum, guar gum such as hydroxypropyl guar, hydrogenated soybean lecithin, sodium alginate, polyacrylamidomethylpropanesulfonic acid, carbomer, cellulose, sodium polyacrylate, konjac gum, agar, and Caesalpinia spinosa gum; more particularly, according to one embodiment of the invention, the composition is free of (0% by weight relative to the total weight of the composition) carrageenan, gellan gum, scleroglucan gum, gum arabic, pectin, xanthan gum, guar gum such as hydroxypropyl guar, hydrogenated soybean lecithin, sodium alginate, polyacrylamidomethylpropanesulfonic acid, carbomer, cellulose, sodium polyacrylate, konjac gum, agar and Caesalpinia spinosa gum.

According to a particular embodiment, a composition according to the invention comprises an amount of less than 0.5% in total amount by weight, relative to the total weight of the composition, of anionic surfactant; more particularly, the composition is free of anionic surfactant (0% by weight relative to the total weight of the composition).

According to a particular embodiment, a composition according to the invention comprises an amount of less than or equal to 0.5% in total amount by weight, relative to the total weight of the composition, of cationic surfactant; more particularly, the composition is free of cationic surfactant (0% by weight relative to the total weight of the composition).

According to a particular embodiment, a composition according to the invention comprises an amount of less than or equal to 0.5% in total amount by weight, relative to the total weight of the composition, of amphoteric surfactant; more particularly, the composition is free of amphoteric surfactant (0% by weight relative to the total weight of the composition).

According to a particular embodiment, a composition according to the invention comprises an amount of less than or equal to 0.5% in total amount by weight, relative to the total weight of the composition, of zwitterionic surfactant, and more particularly is free of zwitterionic surfactant (0% by weight relative to the total weight of the composition).

According to a particular embodiment, a composition according to the invention comprises an amount of less than 0.5% in total amount by weight, relative to the total weight of the composition, of anionic, cationic, amphoteric and zwitterionic surfactant, more particularly an amount of less than 0.10% in total amount by weight, relative to the total weight of the composition, of anionic, cationic, amphoteric and zwitterionic surfactants, even more particularly an amount of less than 0.01% in total amount by weight, relative to the total weight of the composition, of anionic, cationic, amphoteric and zwitterionic surfactants; better still, according to a particular embodiment, the composition is free of anionic, cationic, amphoteric and zwitterionic surfactants (0% by weight relative to the total weight of the composition).

According to a particular embodiment, a composition according to the invention comprises less than 0.5% in total amount by weight, relative to the total weight of the composition, of glyceryl stearate citrate, alkyl sulfate such as sodium lauryl sulfate, alkyl ether sulfate such as sodium laureth sulfate, disodium cocoamphodiacetate, fatty acid polyglyceryl esters such as polyglyceryl-4 isostearate, polyglyceryl-4 diisostearate polyhydroxystearate sebacate, and sodium stearate, glyceryl stearate citrate, alkyl sulfate such as sodium lauryl sulfate, alkyl ether sulfate such as sodium laureth sulfate, disodium cocoamphodiacetate, fatty acid polyglyceryl esters such as polyglyceryl-4 isostearate and polyglyceryl-4 diisostearate polyhydroxystearate sebacate, and sodium stearate.

According to a particular embodiment, a composition according to the invention comprises an amount of less than 1% by weight, relative to the total weight of the composition, of kaolin, perlite, titanium dioxide, talc, cellulose, boron nitride, maltodextrin and mica, and more particularly comprises an amount of less than 0.5% by weight, relative to the total weight of the composition, of kaolin, perlite, titanium dioxide, talc, cellulose, boron nitride, maltodextrin and mica, or even is free of (0% by weight relative to the total weight of the composition) kaolin, perlite, titanium dioxide, talc, cellulose, boron nitride, maltodextrin and mica.

According to a particular embodiment, a composition according to the invention comprises an amount of less than 0.1% by weight, relative to the total weight of the composition, of fatty acid that is solid at room temperature (25 °C), and in particular does not comprise any stearic acid; of benzoic acid, and/or salts thereof, particularly alkali metal or alkaline-earth metal benzoates such as sodium benzoate, of sorbic acid and/or salts thereof, particularly alkali metal or alkaline-earth metal sorbate, including potassium sorbate; of carrageenan, gellan gum, scleroglucan gum, gum arabic, pectin, xanthan gum, guar gum such as hydroxypropyl guar, hydrogenated soybean lecithin, sodium alginate, polyacrylamidomethylpropanesulfonic acid, carbomer, sodium polyacrylate, konjac gum, agar and Caesalpinia spinosa gum; glyceryl stearate citrate, alkyl sulfate such as sodium lauryl sulfate, alkyl ether sulfate such as sodium laureth sulfate, disodium cocoamphodiacetate, fatty acid polyglyceryl esters such as polyglyceryl-4 isostearate and polyglyceryl-4 diisostearate polyhydroxystearate sebacate, and sodium stearate; kaolin, perlite, titanium dioxide, talc, cellulose, boron nitride, maltodextrin and mica.

A composition according to the invention may be in any presentation form normally used in the cosmetics field, for instance in the form of an aqueous gel, lotion, foam, micellar water, cream, paste or serum.

A composition according to the invention is preferentially suitable for topical administration.

Thus, a composition according to the invention may comprise all the constituents usually employed in the envisaged topical application and administration.

A composition according to the invention may advantageously be in the form of an emulsion, particularly obtained by dispersion of an aqueous phase in a fatty phase (W/O) or of a fatty phase in an aqueous phase (O/W), of liquid or semi-liquid consistency of the milk type, or of soft consistency, or even of a multiple emulsion (W/O/W or O/W/O). These compositions are prepared according to the usual known methods.

More particularly, a composition according to the invention may be intended for topical application and may preferably be in the form of an emulsion, preferably an oil-in-water emulsion. Preferably, such an emulsion is not intended to be rinsed off after application.

A composition according to the invention is preferentially intended to be applied to a skin.

Preferably, the skin is the skin of the face, scalp, neckline, neck, arms or forearms, or even more preferably the skin of the face (in particular of the forehead, nose, cheeks and chin), neckline and neck.

The composition may alternatively be in the form of a face and/or body care or makeup product, and may be packaged, for example, in the form of a cream in ajar or a fluid in a tube or a pump bottle or a dropper bottle.

The composition according to the invention may be manufactured via any known process generally used in the cosmetics field.

The ingredients are mixed before forming, in the order and under conditions readily determined by a person skilled in the art.

According to a particular mode of the invention, other agents intended to enhance the appearance and/or texture of the skin may also be added to the composition according to the invention.

### Uses and Processes

According to one of its aspects, the present invention relates to the cosmetic use, particularly the topical use, of a composition according to the invention for preventing and/or treating a skin disorder associated with colonization by bacteria of the species Staphylococcus aureus, and in particular for preventing and/or treating acne and/or eczema in an individual in need thereof.

According to yet another one of its aspects, the present invention relates to a non-therapeutic cosmetic process for caring for keratin materials, in particular the skin, comprising the topical application to these keratin materials of a composition according to the invention.

A skin may in particular be a skin with acne or at risk of having acne and/or a skin with eczema or at risk of having eczema.

The cosmetic uses and processes considered according to the invention are non-therapeutic.

The cosmetic uses and processes of the invention are preferentially performed by topically administering a composition according to the invention.

Topical administration consists of the external application to the skin of cosmetic compositions according to the usual techniques for the use of these compositions.

By way of illustration, the cosmetic use or process according to the invention may be performed by topical, for example daily, application of at least one composition according to the invention, which may be formulated, for example, as a cream, gel, serum, lotion, emulsion or makeup-removing milk.

The application may be repeated for example once or twice daily over a day or more, and generally over an extended period of at least 3 days, at least 4 weeks, or even 4 to 15 weeks, with one or more periods of stoppage, if necessary.

According to one embodiment, the application is daily (once a day) and generally over an extended period of at least 3 days, at least 4 weeks or even 4 to 15 weeks, with one or more periods of stoppage, if necessary.

According to one embodiment, the cosmetic treatment process according to the invention may comprise a single application.

Throughout the description, including the claims, the terms “between . . . and . . . ”, and “ranging from . . . to . . . ” should be understood as including the limit values, unless otherwise specified.

The examples that follow illustrate the present invention without limiting the scope thereof.

In the examples, unless otherwise specified, the temperature is room temperature (25 °C) and is expressed in degrees Celsius, and the pressure is atmospheric pressure.

### Examples

The different compositions tested below are described in Tables 1 to 6 below.

TABLE 1

| | Composition without | Composition with endolysin |
|---|---|---|
| | endolysin of sequence SEQ | of sequence SEQ ID NO: 10 |
| | ID NO: 10 (g per 100 g) | (g per 100 g) |
| Base formula: | qs | qs |
| Preserving | | |
| agent | | |
| Water | | |
| Mineral oil | | |
| Ceteareth 20 | | |
| Endolysin of | — | 0.002565 |
| sequence SEQ | | |
| ID NO: 10 | | |
| Surfactant X | y | y |

These two compositions are compositions according to the invention, comprising at least one surfactant as defined below. Thus, in each of the compositions tested, X is equivalent, respectively, to:

TABLE 2

| | | Nature of | Concentrations |
|---|---|---|---|
| Compositions | X | surfactant | (y) g/100 g |
| 1 | ARACHIDYL ALCOOL 55% | Nonionic | 2 (0.3 of active |
| | (and) BEHENYL ALCOOL | | material) |
| | 30% (and) ARACHIDYL | | |
| | GLUCOSIDE 15% | | |
| | (MONTANOV 202, SEPPIC) | | |
| | (according to the invention) | | |
| 2 | CAPRYLYL/CAPRYL | Nonionic | 2 (1.2 of active |
| | GLUCOSIDE at 60% in water | | material) |
| | (ORAMIX CG 110L SEPPIC) | | |
| | (according to the invention) | | |

TABLE 3

| | Composition without | Composition with endolysin |
|---|---|---|
| | endolysin of sequence SEQ | of sequence SEQ ID NO: 10 |
| | ID NO: 10 (g per 100 g | (g per 100 g) |
| Preserving | 0.2 | 0.2 |
| agent | | |
| Water | qs | qs |
| Mineral oil | 20 | 20 |
| Endolysin of | — | 0.002565 |
| sequence SEQ | | |
| ID NO: 10 | | |
| Surfactant X | y | y |

One of these compositions is a composition according to the invention, comprising at least one surfactant as defined below. A comparative composition, not according to the invention, comprising other surfactants not according to the invention, was also tested. Thus, in each of the compositions tested, X is equivalent, respectively, to:

TABLE 4

| | | Nature of | Concentrations |
|---|---|---|---|
| Compositions | X | surfactant | (y) g/100 g |
| 3 | POLYGLYCERYL-3 | Nonionic | 2 |
| | METHYL GLUCOSE | | |
| | DISTEARATE (TEGOCARE | | |
| | 450 EVONIK | | |
| | GOLDSHIMDT) (according | | |
| | to the invention) | | |
| 4 | POLYGLYCERYL-4 | Nonionic | 2 |
| | ISOSTEARATE (not | | |
| | according to the invention) | | |

TABLE 5

| | Composition without | Composition with endolysin |
|---|---|---|
| | endolysin of sequence SEQ | of sequence SEQ ID NO: 10 |
| | ID NO: 10 (g per 100 g) | (g per 100 g) |
| Preserving | 0.2 | 0.2 |
| agent | | |
| Water | qs | qs |
| Endolysin of | — | 0.002565 |
| sequence SEQ | | |
| ID NO: 10 | | |
| Surfactant X | y | y |

All of these compositions are comparative compositions not according to the invention, and were also tested. Thus, in each of the compositions tested, X is equivalent, respectively, to:

TABLE 6

| | | Nature of | Concentrations |
|---|---|---|---|
| Compositions | X | surfactant | (y) g/100 g |
| 5 | DISODIUM COCOAMPHODIACETATE | Amphoteric | 1.4 (0.4 of active |
| | (not according to the invention) | | material) |
| 6 | SODIUM LAURETH SULFATE (not | Anionic | 10 (7 of active |
| | according to the invention) | | material) |

### Protocol (Experimental Conditions)

Preparation of the working culture of Staphylococcus aureus

Preparation of the working suspension of Staphylococcus aureus (S. aureus) from a lyophilizate ATCC 6538 (according to the recommendations of the standard NF EN 12353). The lyophilizate is rehydrated in Trypticase soy broth, plated on Trypticase soy agar (TSA plates) and incubated for 24 hours at 32.5 °C. The cells are then recovered and resuspended in a commercial cryoprotectant solution with cryobeads for storage at −80 °C for a maximum of 14 months (long-term storage stock).

From a cryobead of this −80 °C stock, subculturing is performed on TSA slant agar which is then incubated for 24 hours at 32.5 °C to obtain the stock culture. This stock culture is stored at 4 °C for a maximum of 9 weeks. The working culture is obtained by subculturing the stock culture onto Trypticase soy agar and incubating it for 24 hours at 35 °C. The working suspension is prepared by suspending the cells of this working culture in a tryptone salt diluent. This suspension is calibrated between 1 and 3×10⁸ CFU (colony forming units)/mL by measuring the absorbance at 620 nm.

Evaluation of the Antimicrobial Activity of the Samples Against S. aureus

The 6 formulations as described above were tested.

At T=1 week of the manufacture of the formulations, 20-gram aliquots of each formulation are inoculated with 0.2 mL of a calibrated suspension of S. aureus. After homogenization, the microorganism content in the product represents a concentration of S. aureus of 10⁶ CFU per gram of product, i.e. a 1% inoculation of a suspension of 10⁸ CFU per mL (the inoculum content is determined by spreading the suspension on Trypticase soy agar plates and incubating for 24 hours at 35 °C). After t=30 minutes and t=60 minutes of contact at room temperature (20 °C±3 °C), 1 gram of the mixture is weighed and 9.0 mL of Eugon LT100 supp broth are then added, followed by mixing until fully homogenized. This mixture is then serially diluted in Eugon LT100 supp broth to 1/100 dilution.

The dilutions are spread on Trypticase soy agar plates and incubated at 35 °C for 48 hours until the surviving colonies of S. aureus are counted.

The antimicrobial activity on S. aureus is expressed in logarithmic abatement relative to the initial content and associated with the activity of the active compound, which is the endolysin of sequence SEQ ID NO: 10 by comparing the counts of surviving S. aureus in formulations with and without the endolysin of sequence SEQ ID NO: 10.

This method is an adaptation of the challenge test method described in the standard “ISO 11930 Cosmetics —Microbiology—Evaluation of the antimicrobial protection of a cosmetic product”.

### Results and Conclusions

The results are expressed as logarithmic Staphylococcus aureus abatement relative to the inoculated content. The abatements are thus between 0 log (no antimicrobial activity observed under the test condition i.e. complete loss of activity of the endolysin of sequence SEQ ID NO: 10) and −5.4 log (maximum reduction observable under the test conditions, i.e. maintenance of endolysin activity under the test conditions). The formula tested is thus particularly active when the values are close to −5.4.

The antimicrobial activity of the endolysin of sequence SEQ ID NO: 10 is evaluated at T=1 week from the date on which the formulations were manufactured. This evaluation is performed according to the protocol described above, i.e. measurement of the activity of the endolysin in the formulation after t=30 minutes of contact and t=1 hour of contact with S. aureus/formulation.

The results are given in Table 7 below.

TABLE 7

| | | Content of | | | |
|---|---|---|---|---|---|
| | | endolysin | | | |
| | Content | of sequence | | Log | Log |
| | surfactant | SEQ ID | | Reduction | Reduction |
| Formulations | X (%) | NO: 10 (%) | pH | t30 min | t1 h |
| 1 ARACHIDYL | 2 | 0 | 6.9 | 0.1 | 0.1 |
| GLUCOSIDE | | 0.002565 | 7.1 | −5.4 | −5.4 |
| 2 | 2 | 0 | 6 | 0.1 | 0.1 |
| CAPRYLYL/CAPRYL | | 0.002565 | 6.2 | −3.7 | −5.4 |
| GLUCOSIDE | | | | | |
| 3 POLYGLYCERYL-3 | 2 | 0 | 8.45 | 0 | 0 |
| METHYL GLUCOSE | | 0.002565 | 7.5 | −1.7 | −2.6 |
| DISTEARATE | | | | | |
| 4 POLYGLYCERYL-4 | 2 | 0 | 6.7 | −0.1 | −0.1 |
| ISOSTEARATE | | 0.002565 | 6.65 | −0.2 | −0.5 |
| 5 DISODIUM | 1.4 | 0 | 5.5 | 0 | −0.2 |
| COCOAMPHODIACETATE | | 0.002565 | 5.6 | 0.1 | −0.1 |
| 6 SODIUM LAURETH | 10 | 0 | 10.3 | 0 | 0 |
| SULFATE | | 0.002565 | 8.7 | 0 | −0.1 |

### Table 7

The results show that the combination of a nonionic surfactant comprising one or more carbohydrate residue(s), such as caprylyl/capryl glucoside, arachidyl glucoside, or polyglyceryl-3 methylglucose distearate, according to the invention with the endolysin of sequence SEQ ID NO: 10 makes it possible to maintain the antibacterial activity against S. aureus of the endolysin—as demonstrated by the Log reduction results in Table 7—compared to the performance obtained with other charged surfactants such as anionic surfactants, amphoteric surfactants and other nonionic surfactants which are free of carbohydrate residue(s).

The results show that the endolysin activity is greater than 99.99% (Log reduction <−4) in destroying S. aureus colonies at 1 week/25 °C for the system comprising caprylyl/capryl glucoside, arachidyl glucoside, or polyglyceryl-3 methylglucose distearate (30 min and 1 h contact time). Caprylyl/capryl glucoside, arachidyl glucoside, or polyglyceryl-3 methylglucose distearate are thus capable of maintaining the antibacterial performance against S. aureus of the endolysin over time, as shown by the Log reduction results in Table 7.

Comparative preparations with the other surfactants not according to the invention tested show significantly lower activity at 1 week under the same conditions.

Surprisingly, the results thus show that the combination of the endolysin with a nonionic surfactant comprising one or more carbohydrate residue(s), such as caprylyl/capryl glucoside, arachidyl glucoside, or polyglyceryl-3 methylglucose distearate, enables better maintenance of the antibacterial activity against S. aureus over time compared to the combination of the endolysin with other charged surfactants such as anionic surfactants, amphoteric surfactants and other nonionic surfactants which are free of carbohydrate residue(s).

Sequence listing
CBD-2638 (PROTEIN)
SEQ ID NO: 1
WKQNKDGIWYKAEHASFTVTAPEGIITRYKGPWTGHPQAGVLQKGQTIKY
DEQKFDGHVWVSWETFEGETVYMPVRTWDAKTGKVGKLWGEIK
CBD-2638 (NUCLEIC ACID)
SEQ ID NO: 2
TGGAAACAGAATAAAGATGGCATTTGGTATAAAGCTGAACATGCTTCGTT
CACAGTGACAGCACCAGAGGGAATTATCACAAGATACAAAGGTCCTTGGA
CTGGTCACCCACAAGCTGGTGTATTACAAAAAGGTCAAACGATTAAATAT
GATGAGGTTCAAAAATTTGACGGTCATGTTTGGGTATCGTGGGAAACGTT
TGAGGGCGAAACTGTATACATGCCGGTACGCACATGGGACGCTAAAACTG
GTAAAGTTGGTAAGTTGTGGGGCGAAATTAAATAA
PLY2638 (PROTEIN)
SEQ ID NO: 3
MLTAIDYLTKKGWKISSDPRTYDGYPKNYGYRNYHENGINYDEFCGGYHR
AFDVYSNETNDVPAVTSGTVIEANDYGNFGGTFVIRDANDNDWIYGHLQR
GSMRFVVGDKVNQGDIIGLQGNSNYYDNPMSVHLHLQLRPKDAKKDEKSQ
VCSGLAMEKYDITNLNAKQDKSKNGSVKELKHIYSNHIKGNKITAPKPSI
QGVVIHNDYGSMTPSQYLPWLYARENNGTHVNGWASVYANRNEVLWYHPT
DYVEWHCGNQWANANLIGFEVCESYPGRISDKLFLENEEATLKVAADVMK
SYGLPVNRNTVRLHNEFFGTSCPHRSWDLHVGKGEPYTTTNINKMKDYFI
KRIKHYYDGGKLEVSKAATIKQSDVKQEVKKQEAKQIVKATDWKQNKDGI
WYKAEHASFTVTAPEGIITRYKGPWTGHPQAGVLQKGQTIKYDEVQKFDG
HVWVSWETFEGETVYMPVRTWDAKTGKVGKLWGEIK
PLY2638 (PROTEIN)
SEQ ID NO: 4
MRGSHHHHHHGSMLTAIDYLTKKGWKISSDPRTYDGYPKNYGYRNYHENG
INYDEFCGGYHRAFDVYSNETNDVPAVTSGTVIEANDYGNFGGTFVIRDA
NDNDWIYGHLQRGSMRFVVGDKVNQGDIIGLQGNSNYYDNPMSVHLHLQL
RPKDAKKDEKSQVCSGLAMEKYDITNLNAKQDKSKNGSVKELKHIYSNHI
KGNKITAPKPSIQGVVIHNDYGSMTPSQYLPWLYARENNGTHVNGWASVY
ANRNEVLWYHPTDYVEWHCGNQWANANLIGFEVCESYPGRISDKLFLENE
EATLKVAADVMKSYGLPVNRNTVRLHNEFFGTSCPHRSWDLHVGKGEPYT
TTNINKMKDYFIKRIKHYYDGGKLEVSKAATIKQSDVKQEVKKQEAKQIV
KATDWKQNKDGIWYKAEHASFTVTAPEGIITRYKGPWTGHPQAGVLQKGQ
TIKYDEVQKFDGHVWVSWETFEGETVYMPVRTWDAKTGKVGKLWGEIK
PLY2638 (NUCLEIC ACID)
SEQ ID NO: 5
ATGCTAACTGCTATTGACTATCTTACGAAAAAAGGTTGGAAAATATCATC
TGACCCTCGCACTTACGATGGTTACCCTAAAAACTACGGCTACAGAAATT
ACCATGAAAACGGCATTAATTATGATGAGTTTTGTGGTGGTTATCATAGA
GCTTTTGATGTTTACAGTAACGAAACTAACGACGTGCCTGCTGTTACTAG
CGGAACAGTTATTGAAGCAAACGATTACGGTAATTTTGGTGGTACATTCG
TTATTAGAGACGCTAACGATAACGATTGGATATATGGGCATCTACAACGT
GGCTCAATGCGATTTGTTGTAGGCGACAAAGTCAATCAAGGTGACATTAT
TGGTTTACAAGGTAATAGCAACTATTACGACAATCCTATGAGTGTACATT
TACATTTACAATTACGCCCTAAAGACGCAAAGAAAGATGAAAAATCACAA
GTATGTAGTGGTTTGGCTATGGAAAAATATGACATTACAAATTTAAATGC
TAAACAAGATAAATCAAAGAATGGGAGCGTGAAAGAGTTGAAACATATCT
ATTCAAACCATATTAAAGGTAACAAGATTACAGCACCAAAACCTAGTATT
CAAGGTGTGGTCATCCACAATGATTATGGTAGTATGACACCTAGTCAATA
CTTACCATGGTTATATGCACGTGAGAATAACGGTACACACGTTAACGGTT
GGGCTAGTGTTTATGCAAATAGAAACGAAGTGCTTTGGTATCATCCGACA
GACTACGTAGAGTGGCATTGTGGTAATCAATGGGCAAATGCTAACTTAAT
CGGATTTGAAGTGTGTGAGTCGTATCCTGGTAGAATCTCGGACAAATTAT
TCTTAGAAAATGAAGAAGCGACATTGAAAGTAGCTGCGGATGTGATGAAG
TCGTACGGATTACCAGTTAATCGCAACACTGTACGTCTGCATAACGAATT
CTTCGGAACTTCTTGTCCACATCGTTCGTGGGACTTGCATGTTGGCAAAG
GTGAGCCTTACACAACTACTAATATTAATAAAATGAAAGACTACTTCATC
AAACGCATCAAACATTATTATGACGGTGGAAAGCTAGAAGTAAGCAAAGC
AGCAACTATCAAACAATCTGACGTTAAGCAAGAAGTTAAAAAGCAAGAAG
CAAAACAAATTGTGAAAGCAACAGATTGGAAACAGAATAAAGATGGCATT
TGGTATAAAGCTGAACATGCTTCGTTCACAGTGACAGCACCAGAGGGAAT
TATCACAAGATACAAAGGTCCTTGGACTGGTCACCCACAAGCTGGTGTAT
TACAAAAAGGTCAAACGATTAAATATGATGAGGTTCAAAAATTTGACGGT
CATGTTTGGGTATCGTGGGAAACGTTTGAGGGCGAAACTGTATACATGCC
GGTACGCACATGGGACGCTAAAACTGGTAAAGTTGGTAAGTTGTGGGGCG
AAATTAAATAA
M23-LST (PROTEIN)
SEQ ID NO: 6
AATHEHSAQWLNNYKKGYGYGPYPLGINGGMHYGVDFFMNIGTPVKAISS
GKIVEAGWSNYGGGNQIGLIENDGVHRQWYMHLSKYNVKVGDYVKAGQII
GWSGSTGYSTAPHLHFQRMVNSFSNSTAQDPMPFLKSAGY
M23-LST (NUCLEIC ACID)
SEQ ID NO: 7
GCTGCAACACATGAACATTCAGCACAATGGTTGAATAATTACAAAAAAGG
ATATGGTTACGGTCCTTATCCATTAGGTATAAATGGCGGTATGCACTACG
GAGTTGATTTTTTTATGAATATTGGAACACCAGTAAAAGCTATTTCAAGC
GGAAAAATAGTTGAAGCTGGTTGGAGTAATTACGGAGGAGGTAATCAAAT
AGGTCTTATTGAAAATGATGGAGTGCATAGACAATGGTATATGCATCTAA
GTAAATATAATGTTAAAGTAGGAGATTATGTCAAAGCTGGTCAAATAATC
GGTTGGTCTGGAAGCACTGGTTATTCTACAGCACCACATTTACACTTCCA
AAGAATGGTTAATTCATTTTCAAATTCAACTGCCCAAGATCCAATGCCTT
TCTTAAAGAGCGCAGGATAT
AMI-2638 (PROTEIN)
SEQ ID NO: 8
NKITAPKPSIQGVVIHNDYGSMTPSQYLPWLYARENNGTHVNGWASVYAN
RNEVLWYHPTDYVEWHCGNQWANANLIGFEVCESYPGRISDKLFLENEEA
TLKVAADVMKSYGLPVNRNTVRLHNEFFGTSCPHRSWDLHVGKGEPYTTT
NINKMKDYFIKRIKHYYDG
AMI-2638 (NUCLEIC ACID)
SEQ ID NO: 9
GGTAACAAGATTACAGCACCAAAACCTAGTATTCAAGGTGTGGTCATCCA
CAATGATTATGGTAGTATGACACCTAGTCAATACTTACCATGGTTATATG
CACGTGAGAATAACGGTACACACGTTAACGGTTGGGCTAGTGTTTATGCA
AATAGAAACGAAGTGCTTTGGTATCATCCGACAGACTACGTAGAGTGGCA
TTGTGGTAATCAATGGGCAAATGCTAACTTAATCGGATTTGAAGTGTGTG
AGTCGTATCCTGGTAGAATCTCGGACAAATTATTCTTAGAAAATGAAGAA
GCGACATTGAAAGTAGCTGCGGATGTGATGAAGTCGTACGGATTACCAGT
TAATCGCAACACTGTACGTCTGCATAACGAATTCTTCGGAACTTCTTGTC
CACATCGTTCGTGGGACTTGCATGTTGGCAAAGGTGAGCCTTACACAACT
ACTAATATTAATAAAATGAAAGACTACTTCATCAAACGCATCAAACATTA
TTATGACGGT
M23-LST AMI2638 CBD2638 (PROTEIN)
SEQ ID NO: 10
AATHEHSAQWLNNYKKGYGYGPYPLGINGGMHYGVDFFMNIGTPVKAISS
GKIVEAGWSNYGGGNQIGLIENDGVHRQWYMHLSKYNVKVGDYVKAGQII
GWSGSTGYSTAPHLHFQRMVNSFSNSTAQDPMPFLKSAGYGKAGGTVTPT
PNTGELLRPKDAKKDEKSQVCSGLAMEKYDITNLNAKQDKSKNGSVKELK
HIYSNHIKGNKITAPKPSIQGVVIHNDYGSMTPSQYLPWLYARENNGTHV
NGWASVYANRNEVLWYHPTDYVEWHCGNQWANANLIGFEVCESYPGRISD
KLFLENEEATLKVAADVMKSYGLPVNRNTVRLHNEFFGTSCPHRSWDLHV
GKGEPYTTTNINKMKDYFIKRIKHYYDGGKLEVSKAATIKQSDVKQEVKK
QEAKQIVKATDWKQNKDGIWYKAEHASFTVTAPEGIITRYKGPWTGHPQA
GVLQKGQTIKYDEVQKFDGHVWVSWETFEGETVYMPVRTWDAKTGKVGKL
WGEIK
M23-LST AMI2638 CBD2638 (PROTEIN)
SEQ ID NO: 11
MRGSHHHHHHGSAATHEHSAQWLNNYKKGYGYGPYPLGINGGMHYGVDFF
MNIGTPVKAISSGKIVEAGWSNYGGGNQIGLIENDGVHRQWYMHLSKYNV
KVGDYVKAGQIIGWSGSTGYSTAPHLHFQRMVNSFSNSTAQDPMPFLKSA
GYGKAGGTVTPTPNTGELLRPKDAKKDEKSQVCSGLAMEKYDITNLNAKQ
DKSKNGSVKELKHIYSNHIKGNKITAPKPSIQGVVIHNDYGSMTPSQYLP
WLYARENNGTHVNGWASVYANRNEVLWYHPTDYVEWHCGNQWANANLIGF
EVCESYPGRISDKLFLENEEATLKVAADVMKSYGLPVNRNTVRLHNEFFG
TSCPHRSWDLHVGKGEPYTTTNINKMKDYFIKRIKHYYDGGKLEVSKAAT
IKQSDVKQEVKKQEAKQIVKATDWKQNKDGIWYKAEHASFTVTAPEGIIT
RYKGPWTGHPQAGVLQKGQTIKYDEVQKFDGHVWVSWETFEGETVYMPVR
TWDAKTGKVGKLWGEIK
M23-LST AMI2638 CBD2638 (NUCLEIC ACID)
SEQ ID NO: 12
GCTGCAACACATGAACATTCAGCACAATGGTTGAATAATTACAAAAAAGG
ATATGGTTACGGTCCTTATCCATTAGGTATAAATGGCGGTATGCACTACG
GAGTTGATTTTTTTATGAATATTGGAACACCAGTAAAAGCTATTTCAAGC
GGAAAAATAGTTGAAGCTGGTTGGAGTAATTACGGAGGAGGTAATCAAAT
AGGTCTTATTGAAAATGATGGAGTGCATAGACAATGGTATATGCATCTAA
GTAAATATAATGTTAAAGTAGGAGATTATGTCAAAGCTGGTCAAATAATC
GGTTGGTCTGGAAGCACTGGTTATTCTACAGCACCACATTTACACTTCCA
AAGAATGGTTAATTCATTTTCAAATTCAACTGCCCAAGATCCAATGCCTT
TCTTAAAGAGCGCAGGATATGGAAAAGCAGGTGGTACAGTAACTCCAACG
CCGAATACAGGTGAGCTCTTACGCCCTAAAGACGCAAAGAAAGATGAAAA
ATCACAAGTATGTAGTGGTTTGGCTATGGAAAAATATGACATTACAAATT
TAAATGCTAAACAAGATAAATCAAAGAATGGGAGCGTGAAAGAGTTGAAA
CATATCTATTCAAACCATATTAAAGGTAACAAGATTACAGCACCAAAACC
TAGTATTCAAGGTGTGGTCATCCACAATGATTATGGTAGTATGACACCTA
GTCAATACTTACCATGGTTATATGCACGTGAGAATAACGGTACACACGTT
AACGGTTGGGCTAGTGTTTATGCAAATAGAAACGAAGTGCTTTGGTATCA
TCCGACAGACTACGTAGAGTGGCATTGTGGTAATCAATGGGCAAATGCTA
ACTTAATCGGATTTGAAGTGTGTGAGTCGTATCCTGGTAGAATCTCGGAC
AAATTATTCTTAGAAAATGAAGAAGCGACATTGAAAGTAGCTGCGGATGT
GATGAAGTCGTACGGATTACCAGTTAATCGCAACACTGTACGTCTGCATA
ACGAATTCTTCGGAACTTCTTGTCCACATCGTTCGTGGGACTTGCATGTT
GGCAAAGGTGAGCCTTACACAACTACTAATATTAATAAAATGAAAGACTA
CTTCATCAAACGCATCAAACATTATTATGACGGTGGAAAGCTAGAAGTAA
GCAAAGCAGCAACTATCAAACAATCTGACGTTAAGCAAGAAGTTAAAAAG
CAAGAAGCAAAACAAATTGTGAAAGCAACAGATTGGAAACAGAATAAAGA
TGGCATTTGGTATAAAGCTGAACATGCTTCGTTCACAGTGACAGCACCAG
AGGGAATTATCACAAGATACAAAGGTCCTTGGACTGGTCACCCACAAGCT
GGTGTATTACAAAAAGGTCAAACGATTAAATATGATGAGGTTCAAAAATT
TGACGGTCATGTTTGGGTATCGTGGGAAACGTTTGAGGGCGAAACTGTAT
ACATGCCGGTACGCACATGGGACGCTAAAACTGGTAAAGTTGGTAAGTTG
TGGGGCGAAATTAAATAA.
6XHIS N-TERMINAL TAG
SEQ ID NO: 13
MRGSHHHHHHGS

1. A composition comprising, in a physiologically acceptable medium:
(i) at least one endolysin; and
(ii) at least one nonionic surfactant comprising one or more carbohydrate residue(s).

2. The composition according to claim 1, wherein the endolysin is an endolysin derived from a Staphylococcus aureus phage.

3. The composition according to claim 1, wherein the endolysin comprises a first protein sequence comprising a domain for binding to the cell wall of species of the genus Staphylococcus.

4. The composition according to claim 3, wherein the first protein sequence is derived from the endolysin of the Φ2638a bacteriophage of S. aureus.

5. The composition according to claim 3, wherein the first protein sequence comprises a protein sequence comprising at least 80% sequence identity with the amino acid sequence of reference SEQ ID NO: 1.

6. The composition according to claim 1, wherein the endolysin comprises a protein sequence comprising at least 80% sequence identity with an amino acid sequence selected from the group consisting of the amino acid sequences of references SEQ ID NO: 3 and SEQ ID NO: 4.

7. The composition according to claim 3, wherein said endolysin also comprises a heterologous protein sequence.

8. The composition according to claim 7, wherein the heterologous protein sequence comprises a lytic domain, said lytic domain comprising a second and a third protein sequence, said second protein sequence comprising an M23 endopeptidase domain and said third protein sequence comprising an amidase domain.

9. The composition according to claim 8, wherein said second and third protein sequences are derived, independently of each other, from an enzyme selected from the group consisting of the endolysin of the Φ2638a bacteriophage of S. aureus and the lysostaphin of S. simulans.

10. The composition according to claim 8, wherein said second protein sequence comprises at least 80% sequence identity with the amino acid sequence of reference SEQ ID NO: 6 and said third protein sequence comprises at least 80% sequence identity with the amino acid sequence of reference SEQ ID NO: 8.

11. The composition according to claim 1, wherein the endolysin comprises a protein sequence comprising at least 80%, sequence identity with an amino acid sequence selected from the group consisting of the amino acid sequences of references SEQ ID NO: 10 and SEQ ID NO: 11.

12. The composition according to claim 1, wherein the endolysin is present in a content ranging from 0.0001% to 0.1% by weight relative to the total weight of the composition.

13. The composition according to claim 1, wherein the nonionic surfactant(s) comprising one or more carbohydrate residue(s) is (are) selected from: i) esters of sugar(s) and fatty acid(s) ii) ethers of sugar(s) and fatty alcohol(s); and iii) mixtures thereof.

14. The composition according to claim 1, wherein the nonionic surfactant(s) comprising one or more carbohydrate residue(s) is (are) selected from alkyl(poly)glycosides and fatty acid (poly)glyceryl esters of glucose or alkylglucose having a linear or branched, saturated or unsaturated, C6-22 hydrocarbon chain, and mixtures thereof.

15. The composition according to claim 13, wherein the nonionic alkyl(poly)glycoside surfactant is represented by the following general formula:
R¹O—(R²O)t−(G)v
wherein:
R¹ represents a linear or branched alkyl or alkenyl radical including 6 to 24 carbon atoms, or an alkylphenyl radical of which the linear or branched alkyl radical includes 6 to 24 carbon atoms;
R² represents an alkylene radical including 2 to 4 carbon atoms;
G represents a sugar unit including 5 to 6 carbon atoms;
t denotes a value ranging from 0 to 10;
v denotes a value ranging from 1 to 15.

16. The composition according to claim 13, wherein the alkyl(poly)glycoside surfactant is selected from caprylyl/capryl glucoside, arachidyl glucoside, and mixtures thereof.

17. The composition according to claim 13, wherein the (poly)glycerol esters of glucose or alkylglucose and of fatty acid surfactant has a linear and saturated C6-22 hydrocarbon chain.

18. The composition according to claim 13, wherein the (poly)glycerol esters of glucose or alkylglucose and of fatty acid surfactant is polyglyceryl-3 methylglucose distearate.

19. The composition according to claim 1, wherein the nonionic surfactant(s) comprising one or more carbohydrate residue(s) are present in a content ranging from 0.01 to 10% by weight relative to the total weight of the composition.

20. The composition according to claim 1, the composition being suitable for topical administration.

21. The composition according to claim 1, for use thereof for preventing and/or treating a skin disorder associated with colonization by bacteria of the species Staphylococcus aureus in an individual in need thereof.

22. The composition for use thereof according to claim 21, wherein the composition is suitable for topical administration.

23. A non-therapeutic cosmetic process for caring for keratin materials comprising the topical application to these keratin materials of a composition according to claim 1.
